(19) United States
(12) Patent Application Publication
(10) Pub. No.: US 2024/0233873 A9
(43) Pub. Date: Jul. 11, 2024

(54) SEQUENCING POLYCLONAL ANTIBODIES DIRECTLY FROM SINGLE PARTICLE CRYOEM DATA

(71) Applicant: THE SCRIPPS RESEARCH INSTITUTE, La Jolla, CA (US)

(72) Inventors: Andrew B. Ward, San Diego, CA (US); Aleksandar Antanasijevic, San Diego, CA (US); Charles Bowman, San Diego, CA (US); Christopher Cottrell, Poway, CA (US); Gabriel Ozorowski, San Diego, CA (US); Robert Kirchdoerfer, Fitchburg, WI (US)

(21) Appl. No.: 18/547,705

(22) PCT Filed: Feb. 24, 2022
(86) PCT No.: PCT/US22/17756

§ 371 Date: Aug. 24, 2023

Related U.S. Application Data
(60) Provisional application No. 63/154,447, filed on Feb. 26, 2021.

(51) Int. Cl. G16B 30/10; G01N 33/577

(52) U.S. Cl. CPC G16B 30/10; G01N 33/577

(57) ABSTRACT

Provided herein are methods for discovery of epitope specific monoclonal antibodies to pathogens directly from immune sera for immunotherapeutic use. Further provided herein are methods to determine molecular structure of antibodies targeting an antigen from convalescent or vaccinated individuals for the purpose of rational vaccine design.

This application claims priority to our co-pending US provisional application with the Ser. No. 63/154,447, filed Feb. 26, 2021, the contents of which is incorporated by reference in its entirety.

### STATEMENT REGARDING FEDERALLY SPONSORED RESEARCH OR DEVELOPMENT

This invention was made with government support under grant nos.: UM1AI144462 and UM1AI100663 awarded by the National Institute of Allergy and Infectious Diseases (NIAID). The United States government has certain rights in the invention.

### FIELD OF THE INVENTION

The present disclosure relates to the fields of identifying antibodies using Cryogenic electron microscopy (CryoEM) and in silico analysis.

### BACKGROUND OF THE INVENTION

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Traditional methods for antibody isolation are based on extensive B-cell sorting that typically require fluorescently labeled probes and access to advanced cell sorting equipment. The sequences of underlying monoclonal antibodies (mAbs) are determined for each individual B cell. These antibodies are subsequently expressed, purified and subjected to further binding, structural and functional evaluation to assess epitope specificity, affinity, and activity (i.e. neutralization capacity).

The traditional methods suffer from a variety of disadvantages. For instance, as the number of isolated monoclonal antibodies can be quite high (>1000), it takes considerable time and reagent requirements to complete the analysis for each antibody. Furthermore, another disadvantage of the current methods is that rational vaccine design methods focus on antibodies that target a certain epitope, that comprise only a small subset of antigen-specific antibodies. High-resolution structural characterization of selected antibodies (alone or in complex with the antigen) is most commonly performed at the end, and it requires to collect a separate dataset (cryoEM or x-ray) for each unique sample. In other words, the current methods do not provide a way to efficiently characterize each and every antibody that binds to the various epitopes in the antigen.

Thus, there remains a need for methods to efficiently analyze and identify monoclonal antibodies.

### SUMMARY OF THE INVENTION

The inventors have now disclosed techniques for sequencing polyclonal antibodies directly from single particle cryogenic electron microscopic (cryoEM data). In one embodiment, disclosed herein is a method of identifying monoclonal antibodies from polyclonal sera. The method utilizes cryogenic electron microscopy-based polyclonal epitope mapping (cryoEMPEM) to determine structural information of a plurality of antigen-antibody complexes in the polyclonal sera, to thereby assign the heavy chain (HC) and light chains (LC), and to identify complementarity determining regions (CDRs). Sequences of polyclonal serum derived antibodies bound to the antigen are then predicted or projected from cryoEM density maps. Clonal family members of the projected antibodies are identified by superimposing cryoEM density maps with sequence and structural data in next generation sequencing (NGS) database. Finally, the identified monoclonal antibodies corresponding to the identified clonal family members are synthesized. In one embodiment, the method may further comprise verifying that the synthesized monoclonal antibodies interact with the antigen in a manner equivalent to the corresponding polyclonal antibodies.

In one embodiment, the clonal family members are further identified by user-defined sequence assignments as a series of numbers. In one embodiment, enzyme-linked immunosorbent assay (ELISA) and biolayer interferometry (BLI) are used to confirm binding of the antibody to the antigen. In one embodiment, the resolution of the cryoEMPEM map is about 3-4 Å. In one embodiment, the identification of clonal family members by further comprises categorizing amino acids in the projected antibodies based on common structural features. Preferably, clonal family members are identified in silico by a sequence alignment and scoring program comprising the steps of: receiving HC and LC repertoires from NGS database, receiving CDR/FR segment assignment from structure based sequence assignment, filtering the HC and LC repertoires and CDR/FR sequence assignment based on CDR length, aligning the filtered NGS sequences to structure-based sequence assignments, and scoring the aligned NGS sequences based on the agreement to structural features. The identified monoclonal antibodies may be used for treatment of a disease caused by the antigen. In one embodiment, the disease is a bacterial disease. In one embodiment, the disease is a viral disease. In one embodiment, the disease is cancer.

In one embodiment, disclosed herein is a method of determining monoclonal antibody sequence from cryoEMPEM maps and NGS data, comprising utilizing structural information obtained from cryoEMPEM maps and NGS data of B-cell repertoires to identify the most probable combination of heavy and light chain pairs corresponding to the antibody of interest. In preferred embodiments, the structure-based sequence assignments are generated from the cryoEM maps using an algorithm to assign different categories of amino acids at each position in a degenerated fashion. In preferred embodiments, the algorithm performs an alignment of the predicted string of amino-acid category identifiers to the antibody sequences from the NGS database and scores the quality of alignment, to thereby select the highest scorers as the monoclonal antibody sequence. In one embodiment, the method further comprises synthesizing the monoclonal antibodies. In one embodiment, the method further comprises binding the synthesized monoclonal antibody with to the antigen to confirm high-affinity binding. In one embodiment, the categorization of amino acids is based on common structural features.

In one embodiment, disclosed is a method of designing a vaccine for a disease, comprising: obtaining, or having obtained, a sample from the patient; determining structural information of a plurality of antigen-antibody complexes in the patient sample, via cryoEMPEM, to thereby assign the heavy and light chains and to identify complementarity determining regions; analyzing cryoEM density maps in silico to predict possible antibody sequences bound to the antigen; identifying clonal family members of the antibody, by superimposing structural data in cryoEM density maps with sequences in the NGS database; and designing a treatment for the disease by synthesizing the monoclonal antibody corresponding to the identified clonal family members. In one embodiment, the sample is a blood or plasma sample, or a tumor sample. In one embodiment, the disease is a bacterial disease. In one embodiment, the disease is a viral disease. In one embodiment, the disease is cancer.

Various objects, features, aspects, and advantages will become more apparent from the following detailed description of preferred embodiments, along with the accompanying drawing in which like numerals represent like components.

### BRIEF DESCRIPTION OF THE DRAWING

FIG. 1 depicts an exemplary embodiment of the methods disclosed herein. (a) Cartoon illustration showing how cryoEMPEM and NGS data are applied to predict sequences. (b) High resolution maps and corresponding models of immune complexes with polyclonal antibodies (from left to right, Rh.33172 pAbC-2, Rh.33104 pAbC-1, and Rh.4O9 pAbC-1). Full maps and models are shown on the bottom and the close-up view of the epitope-paratope interface is shown on top. Maps are represented as transparent light gray mesh. Ribbon representation of structure models are used (BG505 SOSIP—dark gray, polyclonal Fab backbone models—green and pink). N-linked glycans are shown as sticks and colored yellow. This data is adapted from Antanasijevic et al., 2020 (off-target responses paper). The highly resolved Fab density suggests that the compositional heterogeneity originating from the polyclonal nature of bound Fabs is relatively low.

FIG. 2 depicts exemplary assay and cryoEM mapping as disclosed. (a) Pseudovirus neutralization assay results for Rh.4O9.7 and Rh.4O9.8 antibodies (left) adapted from Zhao et al., 2020. nsEM-based 3D map of the Rh.4O9 8 mAh (as Fab Fragments) in complex with BG505 SOSIP trimer (right). The arrows show the location of the antibody. (b) Overlay of the nsEM map with Rh.4O9.8 mAb (transparent white-gray mesh) and the Rh.4O9 pAbC-1 cryoEM map (BG505 SOSIP—gray; polyclonal antibody—green). (c) Ribbon representation of the atomic model of BG505 SOSIP (peptide—dark gray; glycans—yellow) and Rh.4O9.8 mAb (green) complex built into the polyclonal cryoEMPEM map, Rh.4O9 pAbC-1 (transparent gray mesh). (d) Close-up view showing model-to-map fit for the heavy chain (top) and the light chain (bottom) of the Rh.4O9.8 mAb (heavy chain—olive green; light chain—forest green; EM map—transparent gray mesh).

FIG. 3 depicts exemplary Sequence prediction from high resolution cryo-EMPEM maps. (a) Amino-acid categories were defined based on common structural features. Hierarchical system allows for confidence-based assignment of amino-acids and offers more flexibility. (b) Example of the amino-acid assignment process using Rh.33104 pAbC-1 (heavy chain, residues 55-63). Category assignments are based on the structural features at each amino-acid position. Models are displayed in pink and maps are represented as transparent light-gray surface. The bottom part shows the best matching sequence from the database search and the corresponding model. (c) Flowchart of the steps within the sequence alignment and scoring program. (d) Examples of matching and scoring results for a subset of NGS sequences, calculated from the search algorithm output data (Rh.33104 dataset, light chain query). The parameters are calculated for the entire aligned portion of the sequence (total) and for CDR segments without the framework (CDR-only). For clarity, the category labels are depicted by 1-3 letter abbreviations.

FIG. 4 depicts exemplary characterization of the recovered monoclonal antibodies. (a,d) Results of the BLI binding analysis performed with Rh.33104 mAb.1 (a) Rh.33172 mAb.1 (d) in the form of IgG and the corresponding BG505 SOSIP trimer antigen. VRC01 IgG (gray curves) was used for reference. (b, e) CryoEM maps and models of the BG505 SOSIP complexes with Rh.33104 mAb.1 (b) and Rh.33172 mAb.1 (e) as Fabs. Map is represented as transparent gray mesh and models are shown as ribbons (BG505 SOSIP—dark gray; RM19R and RM20A3—purple; Rh.33104 mAb.1—pink; Rh.33172 mAb.1—green). (c, f) Overlay of the cryoEM maps/models from the EM experiments with monoclonal and polyclonal Fabs for Rh.33104 (c) and Rh.33172 (f) samples. Full maps of immune complexes for comparison are shown on top in each panel. Maps are color-coded for clarity (BG505 SOSIP—light gray; RM20A3 and RM19R—purple; light pink—Rh.33104 mAb.1; deep pink—Rh.33104 pAbC-1; forest green—Rh.33172 mAb.1; olive green—Rh.33172 pAbC-1). Map (left) and model (right) overlays focusing on the Fab are shown in bottom panels. Same color scheme was used as with full maps.

FIG. 5 depicts exemplary comparison of the best-matching sequence to clonal relatives from the NGS database for Rh.33104 mAb.1 (a) and Rh.33172_mAb.1 (b) antibodies. Only CDR sequence alignments are shown. The models of the best-matching sequence and the consensus sequence focusing to the CDR sites where mismatches exist between the two are shown below. Cryo-EMPEM map is represented as transparent gray surface.

FIG. 6 illustrates correlation curves and resolution plots. (a) Fourier shell correlation curves for trimer-pAbC complexes obtained using cryoEMPEM. (b) Local resolution plots for the trimer-pAbC complexes understudy.

FIG. 7 illustrates analysis of the model-to-map fit for the Rh.4O9.8 antibody and the Rh.4O9 pAbC-1 cryoEMPEM map. Per-residue Q-score values for the heavy (top) and light (bottom) chains of the Rh.4O9. Average Q-score for each chain is displayed as dotted red line. CDRs are represented in orange.

FIG. 8 illustrates amino acid assignment tree with numerical category identifiers.

FIG. 9 illustrates the sequence and matching process. (a) Example of the amino-acid assignment for Rh.33104_PAbC.1. For light chain (left), residues 94-102 and the corresponding area of the map are shown. For heavy chain (right), residues 102-110 and the corresponding area of the map are shown. Models are displayed in pink and maps are represented as transparent light-gray surface. (b) Best sequence matches for Rh.33104_PAbC.1 light chain (top) and heavy chain (bottom). Matching to assignments at each position is shown (+/−). Overall agreement to predictions and scoring data are shown on the right.

FIG. 10 illustrates the sequence and matching process. (a) Example of the amino-acid assignment for Rh.33172 pAbC-2. For light chain (left), residues 32-40 and the corresponding area of the map are shown. For heavy chain (right), residues 107-115 and the corresponding area of the map are shown. Models are displayed in green and maps are represented as transparent light-gray surface. (b) Best sequence matches for Rh.33172 pAbC-2 light chain (top) and heavy chain (bottom). Matching to assignments at each position is shown (+/−). Overall agreement to predictions and scoring data are shown on the right.

FIG. 11 show examples of the most common mismatches observed in the Rh.33104 (pink) and Rh.33172 searches (green). (a) Small side-chain, SSC, category (amino acids: A, S, C, T, V) was predicted based on structural data but P/G category (amino acids: P and G) was in the sequence. (b) Small side-chain, SSC, category (amino acids: A, S, C, T, V) was predicted based on structural data but medium side-chain LMC category (amino acids: H, N, D, Q, E) was in the sequence. Map segments are represented as transparent light-gray surface.

FIG. 12 illustrates binding data for Rh.33104 mAb.1 and Rh.33172 mAb. 1. Sandwich ELISA was used to quantify the interaction of BG505 SOSIP to the IgG versions of Rh.33104 mAb.1 (a) and Rh.33172_MAb.1 (c). VRC01 IgG was used as a reference. IC50 values are in the tables below the corresponding graphs. BLI was used to determine the kinetic binding parameters of the interaction between BG505 SOSIP and the Fab versions of Rh.33104 mAb.1 (b) and Rh.33172 mAb.1 (d). Antigen concentrations used to generate each binding curve are illustrated on the right in panels (b) and (d), while the corresponding kinetic parameters are presented in the table below each graph

FIG. 13 illustrates schematic representation of the data processing workflow for cryoEM data with relevant statistics. The samples were (a) BG505 SOSIP complexed with Rh.33104 mAb.1 and RM20A3 and (b) BG505 SOSIP complexed with Rh.33172 mAb.1 and RM19R

FIG. 14 illustrates an overview of the B-cell sorting procedure.

FIG. 15 illustrates cryoEMPEM analysis of OC43 spike-polyclonal Fab complexes. (A) Highresolution cryo-EMPEM reconstructions of OC43 spike complexed with polyclonal Fabs derived from sera from HDs 269 (top-left), 1051 (top-right) or 1412 (bottom); the representative composite figures from ns-EMPEM from these donors are shown in the middle. Each map depicts a structurally unique polyclonal antibody class reconstructed at the indicated resolution with the Fabs. OC43 spike is represented in light gray. Fabs marked with a black dot were observed by ns-EMPEM but were not detected by cryo-EMPEM. Fab class from donor 269 marked with a star was resolved by cryo-EMPEM but not by nsEMPEM. (B) Sapienic acid (aquamarine) binding within a hydrophobic pocket in the CTD-CTD inter-protomeric interface. Protomers are colored in light pink, blue or wheat and the interacting residues are shown in gray.

FIG. 16 illustrates Cryo-EM structures of polyclonal Fabs targeting the OC43 spike (A-G) Tube or ribbon representation of atomic models of OC43 spike-Fab complexes along with zoomed-in views of epitope-paratope interactions. (A-B) Fab1 and Fab2 (red) target the NTD-site1 or RBS, (C) Fab3 (orange) targets NTD-site2 adjacent to RBS, (D-F) Fab4, Fab5 and Fab6 (yellow) target the CTD and (G) Fab? (blue) targets the NTD-CTD interface. The spike protomers are shown in light blue, light pink or wheat (ribbon representation) with glycans in teal (sphere atom representation) and primary epitope contacts in gray. (H) Surface representation of OC43 spike (gray) showing collective epitopes of Fab1 to Fab10 colored based on their binding site

FIG. 17 illustrates Ns- and cryo-EMPEM analysis of polyclonal Fabs from SARS-2 CD sera. (A) Representative 2D classes and side and top views of composite figures from ns-EMPEM analysis of polyclonal Fabs from 3 SARS-2 donors complexed with β-CoV spikes. The donor numbers along with the corresponding CoV spikes are indicated above each panel in (A). The Fabs are color-coded based on their epitope specificities as indicated at the bottom-left. SARS-2, OC43, HKU1 and MERS spikes are represented in slate gray, light gray, dark gray and beige respectively. 3D reconstructions displaying potential self-reactive antibodies are shown in grey on the top right corners for both donors 1988 and donor 1999 in complex with SARS-2 spike (B) Composite figure showing 5 unique antibody classes, Fab11 to Fab15 colored in shades of red, to SARS-2 spike NTD reconstructed using cryo-EMPEM analysis of polyclonal Fabs from donors 1988 and 1989 complexed with SARS-2 stabilized spikes. (C) Surface representation of SARS-2 spike showing epitopes of Fabs 11 to 15 from (B) on a single NTD (slate gray) with a zoomed-in view displaying the loop residues comprising each epitope. Loop 144 to 156 with the N149 glycan forms an immunodominant element commonly targeted by Fabs 11 to 14. The sub-epitope colors correspond to each Fab shown in (B).

FIG. 18 illustrates high-resolution cryoEM defines broadly reactive epitopes of H5N1 vaccine-elicited pAbs. (A) CryoEM-mapped epitopes targeted by pAbs from subject 4 at day 28. RBS epitope, red; lateral patch epitope, yellow; vestigial esterase epitope, pink; VH1-69 epitope, blue. (B) Each epitope specificity marked together on a single trimer. (C and D) CryoEM map of stem immune complex from subject 4 at day 28 with ribbon diagrams for H5 HA (A/Indonesia/5/2005, gray; PDB: 4K62) and mAb 1C01 from subject 4 day 7 docked into the EM density. Full-length side view (C) and zoomed in view of the epitope-paratope interaction (D). VH, blue; VL, green; CDRH2, dark blue; CDRH3, purple; HA residues H18 and W21, red.

FIG. 19 illustrates identification of epitope-specific functional antibodies against membrane proteins by using the cryoEMPEM method disclosed herein (A) Full method workflow. (B) ELISA data acquired for week 6 serum samples from rabbits immunized with Piezol. Different lines indicate different serum samples.

FIG. 20 illustrates the method for determining antibody germline sequences using cryoEMPEM (A) Illustration of the germline sequence inference method used for determination of macaque Vh/Vl sequences. (B) Table summary of the validation run results.

FIG. 21 illustrates In silico method for antibody design using structural data from cryoEMPEM maps (A) Illustration of the method for antibody design using structural restraints from cryoEMPEM maps. (B) Sequence logo of Rosetta-designed models for Rh.4O9 pAbC-1 (only CDR loop sequences are shown). (C) Overlay of the top scoring model (green) and the monoclonal antibody Rh.4O9.8 (salmon). Close-up view of the HCDR3 loop with amino-acids shown. BG505 SOSIP antigen is presented in gray.

FIG. 22 illustrates application of cryoEMPEM for isolation and identification of auto-immune antibodies cryoEMPEM maps of rabbit polyclonal antibodies (colored based on the epitope) bound to HIV Env trimer (gray). Antibodies represented in dark gray (left panel), yellow (middle panel) bind the HIV Env antigen in a manner that is dependent on other antibodies. On the other hand, type-3 antibody in turquoise (right panel) binds to the epitope fully comprised of another antibody presented in dark green and does not interact with the antigen.

FIG. 23 illustrates application of cryoEMPEM for characterization of antibodies against snake venom proteins (A) Electron microscopy analysis of snake venom proteins. Representative 2D class averages are shown on top and the corresponding 3D reconstruction is represented as transparent gray surface. Structures of best matching homologous proteins are fit into each map. (B) 2D class averages of immune complexes featuring different snake venom proteins and polyclonal antibodies from EchiTAbG antivenom.

### DETAILED DESCRIPTION

The inventors have now discovered a method of efficiently sequencing polyclonal antibodies directly from single particle cryoEM data. As disclosed throughout, the approach starts with a high-resolution cryoEMPEM analysis on the serum level. Single cryoEM dataset was collected per sample and elicited polyclonal antibodies in complex with the antigen were reconstructed. The structural information was coupled with the B-cell sequence library obtained through next-generation sequencing (NGS), to identify antibodies bound to different epitopes. Monoclonal antibodies with desired epitope specificities were expressed and subjected to functional characterization. This bypasses the requirement for single B-cell sorting and monoclonal antibody screening. Therefore, the analysis can be completed within a few weeks of sample collection, instead of a few months that the currently available methods require. Additionally, combining the cryoEM and NGS methods in this manner reduces the total cost in equipment and reagents. Finally, this approach eliminates the need for high-resolution characterization of identified monoclonal antibodies as the data is already acquired on the polyclonal level.

One of the problems, or rate-limiting step, of the currently available methods in analyzing immune responses to vaccines or infections, is the isolation and characterization of monoclonal antibodies, because such methods are time consuming, inefficient, and expensive. The instant disclosure provides a solution to this problem—the methods presented herein is a hybrid structural and bioinformatic approach to directly assign the heavy and light chains, identify complementarity-determining regions, and predict possible sequences from cryoEM density maps of polyclonal serum derived antibodies bound to an antigen. When combined with next generation sequencing of immune repertoires, the method enables a user to specifically identify clonal family members, synthesize the monoclonal antibodies and confirm that they interact with the antigen in a manner equivalent to the corresponding polyclonal antibodies. This structure-based approach for identification of monoclonal antibodies from polyclonal sera opens new avenues for analysis of immune responses and iterative vaccine design.

Comprehensive analyses of immune responses to infection or vaccination are challenging, laborious and expensive. Classical serology approaches, based on ELISA and viral neutralization assays, offer a wealth of information but require a relatively large set of biological and viral reagents. Increasingly, for rational and structure-based vaccine design efforts it is necessary to go beyond the serum analysis and identify specific monoclonal antibodies elicited by a vaccine/pathogen. Isolation of antigen-specific B-cells, in the most standard form, requires fluorescently labeled antigen probes and access to advanced cell sorting equipment. After B-cell sorting, individual monoclonal antibodies are expressed, purified and subjected to further binding, structural and functional evaluation to identify the antibodies with appropriate affinity, epitope specificity and activity (i.e. neutralization capacity).

Negative stain electron microscopy (nsEM) may be utilized for characterization of polyclonal antibody responses elicited by vaccination of infection (EM-based polyclonal epitope mapping—EMPEM), as disclosed in US Pub. No.: 20190325985, the entire contents of which is incorporated by reference herein, including the drawings. EMPEM typically identifies the most abundant polyclonal antibody classes targeting unique epitopes on the antigen surface. This analysis offers information regarding the immunogenic landscape of a given antigen. Additionally, the structural component of these experiments elucidates the mode of binding for different polyclonal antibodies (e.g. angle of approach, contact surface, comparison to other known antibodies etc.). EMPEM in combination with standard serological approaches (ELISA, neutralization assays) therefore provides a powerful tool for evaluation and comparison of different immunization approaches in a range of animal models (including humans).

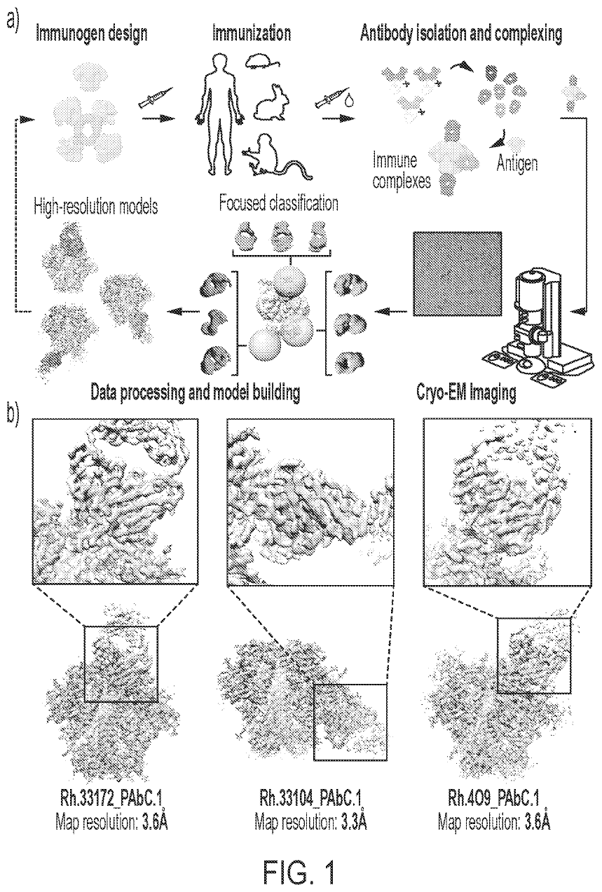

EMPEM can also be conducted under cryo conditions (cryoEMPEM) enabling high-resolution information on the antigen—antibody immune complexes (FIG. 1.a). By acquiring large cryoEM datasets and subjecting the particle projection images to extensive computational sorting we can readily reconstruct maps of immune complexes featuring polyclonal antibodies at 3-4 Å resolution (examples are shown in FIG. 1.b). The polyclonal nature of the bound antibodies and the lack of sequence information has however restricted true atomic resolution of the reconstructed maps, and thereby limited the interpretation of specific epitope-paratope contacts.

The techniques disclosed herein offer a hybrid method for determination of monoclonal antibody sequences directly from cryoEMPEM maps. The method utilizes structural information and next generation sequencing (NGS) data of B-cell repertoires as the main inputs to identify the most probable combination of heavy and light chain pairs corresponding to the antibody of interest. The structure-based sequence assignment algorithm of this disclosure uses different categories of amino acids at each position in a degenerate fashion. The program then performs an alignment of the predicted string of amino-acid category identifiers to the antibody sequences from the database and scores the quality of alignment.

The method was tested on two high resolution cryoEMPEM datasets featuring polyclonal antibodies in complex with stabilized HIV Env trimer antigens (BG505 SOSIP). Predicted monoclonal antibodies were synthesized and the binding to the antigen was confirmed using ELISA and biolayer interferometry (BLI). CryoEM characterization was performed on the two monoclonal antibodies in complex with the corresponding BG505 SOSIP antigen and the reconstructed cryoEM maps and models showed excellent agreement with the cryoEMPEM data used for sequence prediction; confirming that the tested monoclonal antibodies belong to the corresponding polyclonal antibody families elicited by vaccination and validating the approach.

In one embodiment, the presently disclosed methods are complementary to current approaches used for isolation of monoclonal antibodies with a few key advantages. The application of structural information allows a user to select only the polyclonal antibodies against specific epitopes. NGS data can directly be used for sequence analysis of the clonal family of the antibody in question. The new antibody sequence determination method would facilitate the mAb discovery process and accelerate iterative vaccine design.

Viewed from another perspective, the inventors have expanded the range of applicability cryoEMPEM data and introduced a platform to identify functional antibody sequences from structural observations. In doing so, they have circumvented the need to isolate monoclonal antibodies using antigen specific B-cell sorting or functional screening. Further, the currently used methods of B-cell sorting do not necessarily represent the most abundant antibodies at the serum level, while the instantly disclosed cryoEMPEM of sera does so. By directly imaging the serum antibodies, a user is able to obtain a proxy for abundance, affinity, and clonality. This information further complements the NGS data and allows for more extensive sequence analysis.

The currently disclosed approach can reliably assign Vh/Vl genes from a germline database as well as assign CDR loop length, structure, and properties. Further, the structural information can be derived in a matter of days. This allows for a more immediate impact on vaccine design, including real time decision making during immunizations, immunogen redesign for on- and off-target responses, creation of probes for sorting specific B-cell responses. The monoclonal antibodies recovered using this method can potentially be useful as immunotherapeutics, such as those being sought for bacterial, viral, or cancer pathogens, as well as emerging pathogens like SARS-CoV-2, or reagents for other assays. Thus, it provides an alternative to expensive traditional approaches based on B-cell isolation or screening, or generation and selection of hybridomas. The method can be applied to a wide array of antigen-polyclonal antibody samples and should have broad utility in vaccine design and immunotherapeutic development. In one embodiment, the methods disclosed herein may also be fully automated, which further speeds up and simplifies the sequence determination process.

Embodiments of the present disclosure are further described in the following examples. The examples are merely illustrative and do not in any way limit the scope of the invention as claimed.

### EXAMPLES

### Example 1: Antibody Sequence Information is Preserved in cryoEMPEM Maps

To demonstrate the approach, the inventors selected three immune sera elicited in Rhesus macaques following an immunization with BG505 SOSIP-based immunogens from previously described studies (Nogal et al, 2020; Antanasijevic et al., 2020). These three were chosen because each contained structurally unique polyclonal antibody class (pAbC). Further, each resulted in high quality single particle cryoEM reconstructions (3.3-3.6 Å) with high local resolution for the part of the EM map corresponding to the Fab (FIG. 1a,b). We then utilized each reconstruction to optimize our method for directly sequencing the antibody from the reconstructed density.

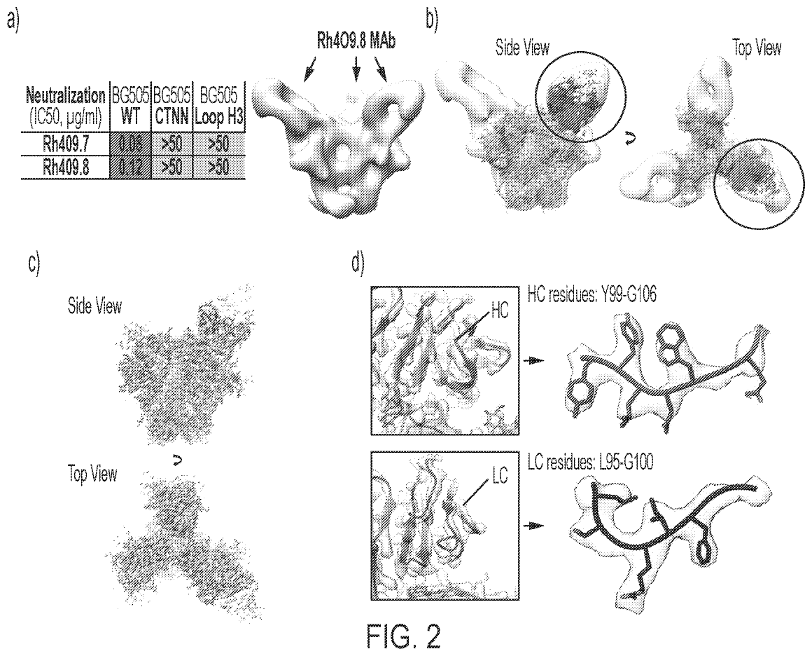

The inventors first analyzed the Rh.4O9 pAbC-1 dataset (FIG. 1.b, right), which had an antibody bound to the V1 loop of BG505 SOSIP antigen (Nogal et al., 2020), and for which corresponding monoclonal antibodies had recently been isolated (Zhao et al., 2020). Two of the antibodies from the same clonal family, Rh.4O9.7 and Rh.4O9.8, neutralized the WT BG505 pseudovirus and appeared to target the V1 based on mutagenesis (FIG. 2.a). nsEM reconstruction of Rh.4O9.8 mAb bound to BG505 SOSIP confirmed the V1-specificity (FIG. 2.a) and superimposed with the polyclonal Fab from the Rh.4O9 pAbC-1 map (FIG. 2.b). This suggested that Rh.4O9.8 mAb (and other clonal relatives) likely comprise the polyclonal antibody family reconstructed in Rh.4O9 pAbC-1 map. The sequence of Rh.4O9.8 was therefore used to build an initial atomic model into the Fab-corresponding part of the Rh.4O9 pAbC-1 map (FIG. 2.c). The model exhibited excellent fit at the secondary structure (FIG. 2.d left) as well as side chain level (FIG. 2.d right), including the complementarity determining regions (CDRs). Thus, in this example the mAb isolated at a timepoint is determined as a close relative of the V1 targeting pAb identified at the serum level by cryoEM.

### Example 2: Hierarchical Approach for Structure-Based Sequence Assignment

In the above example having a monoclonal antibody enabled interpretation of cryoEM density at an atomic level. However, what does one do in the much more typical situation in which monoclonal antibodies have not been generated. Given the polyclonal nature of bound antibodies and final cryoEMPEM map resolutions of ˜3-4 Å, the structural information is too ambiguous to directly assign antibody sequences. However, we reasoned that appropriate sequence database featuring the BCR repertoire at a corresponding time point was available the structural information could be used to identify the heavy and light chain sequence candidates. Thus, we developed a hybrid approach that utilizes the structural information from cryoEMPEM maps and next generation sequencing (NGS) data of B-cell repertoires for identification of monoclonal antibodies.

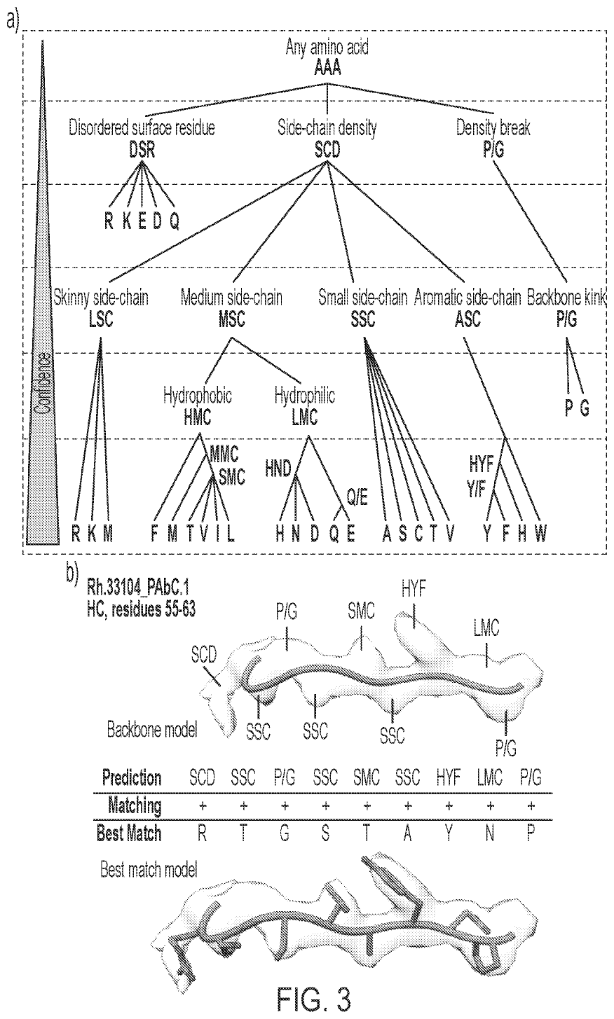

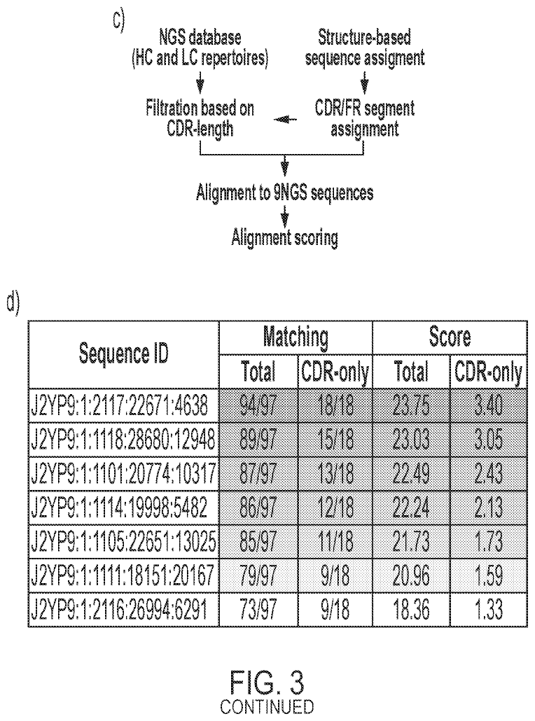

To approximate the ambiguity inherent in interpreting 3-4 Å resolution cryoEM maps we developed an algorithm weights the degree of certainty associated with the corresponding structural features (i.e. density volume surrounding each amino acid) (FIG. 3.a). The assignments are performed manually with each amino acid position depicted with a hierarchical category identifier corresponding to a predefined subset of amino acid residues that best fit the density. An example of category-based assignment using the Rh.33104 pAbC-1 map is shown in FIG. 3.b. The examples of full sequence assignments for the polyclonal antibodies (Rh.33172 pAbC-2 and Rh.33104 pAbC-1) are presented. At the end of this process, structural homology with published antibody structures is used to define the CDR and framework (FW) regions, thereby resulting in an initial hierarchical sequence assignment of the antibody.

Next, we designed a search algorithm to take two main inputs (1) the preprocessed NGS database and (2) the user-defined sequence assignments as a series of numbers (FIG. 3.c). NGS data is pre-filtered by CDR lengths determined during the sequence assignment step by a user-set length tolerance. The program then performs a non-gapped exhaustive alignment search of the query vs every sequence remaining in the database. The queries are split by feature (FW1/2/3, CDR1/2/3) and each feature is aligned independently. Matching at each site is determined based on the agreement of the assigned category and the corresponding amino acid from the aligned NGS sequence. For each match (depicted as “+” in the outputs) at a given position, the score is assigned based on the relative ambiguity of the category (1/Xᵢ where Xᵢ=the number of possible amino acids, X, at position i). The score is not calculated for mismatches (depicted as “-” in the outputs). The output contains the sequences ranked based on the CDR lengths, alignment scores and the number, location and nature of mismatches (if any). Example output files for Rh.33104 and Rh.33172 searches were determined, see the next section for details. Calculated score and matching are the two main parameters used to evaluate the agreement of structural data with the sequences from the database (FIG. 3.d).

### Example 3: Sequence Prediction Platform

Our sequence prediction algorithm was then applied to samples collected from animals Rh.33104 and Rh.33172. The germinal center B-cells were harvested from both animals at the same time point as the cryoEMPEM analysis (Week 27) via fine needle aspiration (FNA). Extracted B-cells were sorted based on their binding to the BG505 SOSIP antigen. The sorted B-cells from Rh.33104 and Rh.33172 were then separately pooled, lysed and subjected to next generation sequencing (NGS), as described in the method section. Notably, the specific heavy and light chain pairing is lost during this process. Before starting the search, the NGS read data was prepared by collapsing duplicate reads and performing analysis on the resulting sequences using IGBLASTn. Sequences designated as non-productive by IGBLASTn are removed from the search pool, and the remaining productive reads are filtered based on feature lengths. For heavy chain queries a single NGS database is used for each antibody. For light chains, the search is performed independently for the Ig-κ and Ig-λ databases.

Structure-based sequence category assignments for Rh.33104 pAbC-1 and Rh.33172 pAbC-2 are provided. The NGS database alignment results with scores were determined. During the alignment analysis, special emphasis was placed on the score within the CDR regions as the most relevant site for comparisons between different antibodies. The heavy and light chain sequence candidates with the highest scores (total and CDR-only) and best matching to predictions were selected and evaluated. Model-to-map fits, alignment and scoring statistics for the best matching Rh.33104 pAbC-1 and Rh.33172 pAbC-2 sequences are calculated.

Structural models of antibodies based on the recovered heavy and light chain sequences exhibited excellent fits into the corresponding pAbC maps. The mismatches (disagreements between the assigned amino acid category and the amino acid from the best matching sequence) comprised 4-18% of the residues in the full heavy and light chain sequences. For CDRs the mismatches comprised 0-14% of the cases. However, based on the structural analysis these discrepancies did not result in a major change in amino acid type at any of the sites. Most commonly, there was a switch between the small side chain, SSC, category (amino acids: A, S, C, T, V) and P/G category (amino acids: P and G) which are often difficult to distinguish; or a change between the SSC category (amino acids: A, S, C, T, V) and the medium side-chain LMC category (amino acids: H, N, D, Q, E).

### Example 4: Validation of the Sequence Prediction Platform

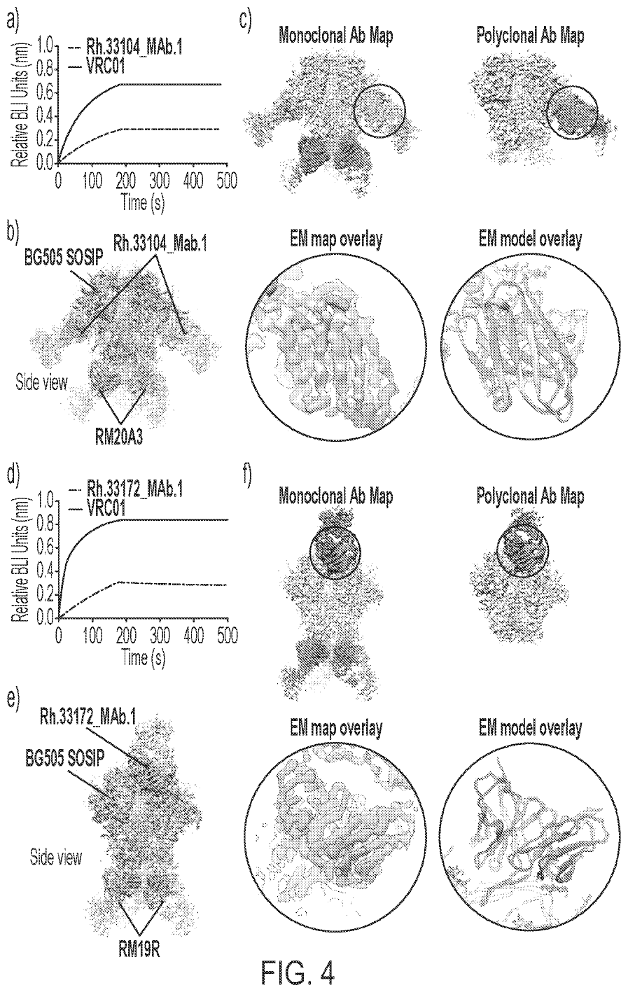

Antibodies based on the top-scoring heavy and light chain sequences from Rh.33104 and Rh.33172 queries were produced and assessed for binding to the BG505 SOSIP antigen using biolayer interferometry (BLI) and sandwich ELISA assays (FIG. 4.). Notably, both monoclonal antibodies, termed Rh.33104 mAb.1 and Rh.33172 mAb.1, formed functional dimeric IgG and bound BG505 SOSIP as IgG (FIG. 4.a,d) and Fas. IC₅₀ values from ELISA experiments with IgGs were 1.93 μg/ml and 2.64 μg/ml and the dissociation constants (K_{d}) from BLI were 890 nM and 180 nM, for Rh.33104 mAb.1 and Rh.33172 mAb.1, respectively. These binding affinities are comparable to other mAbs isolated from Rhesus macaques in published immunization studies (Cottrell et al., 2020).

For further validation the two antibodies (as Fabs) were independently complexed with BG505 SOSIP antigen and subjected to cryoEM analysis. The final map resolutions for the Rh.33104 mAb.1- and Rh.33172 mAb.1-containing complexes were 3.3 Å and 3.5 Å, respectively. Atomic models were relaxed into the reconstructed maps (FIG. 4. b and e). The structures revealed that both antibodies bound to the same epitopes on BG505 SOSIP surface as the polyclonal antibodies computationally sorted from cryoEMPEM maps (FIG. 4. c and f). The overlay of the maps/models from experiments with monoclonal and polyclonal Fabs revealed excellent agreement in both cases (FIG. 4. c and f, bottom). On the backbone level (Ca), the RMSD values between the monoclonal and polyclonal Ab models were 0.668 Å and 0.721 Å for Rh.33104 and Rh.33172 datasets, respectively. Altogether, these findings confirmed that the monoclonal antibodies selected from the NGS database were clonal members of the polyclonal families detected via cryoEMPEM.

### Example 5: Sequence Analysis and Comparison to the Structural Data

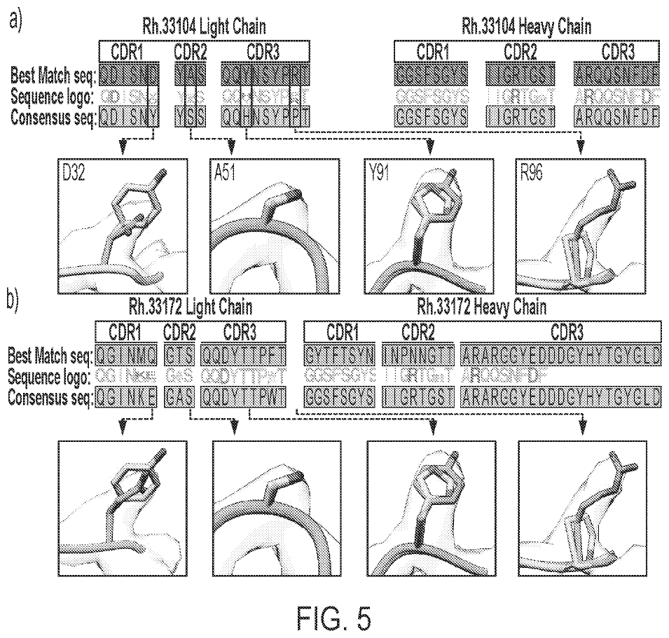

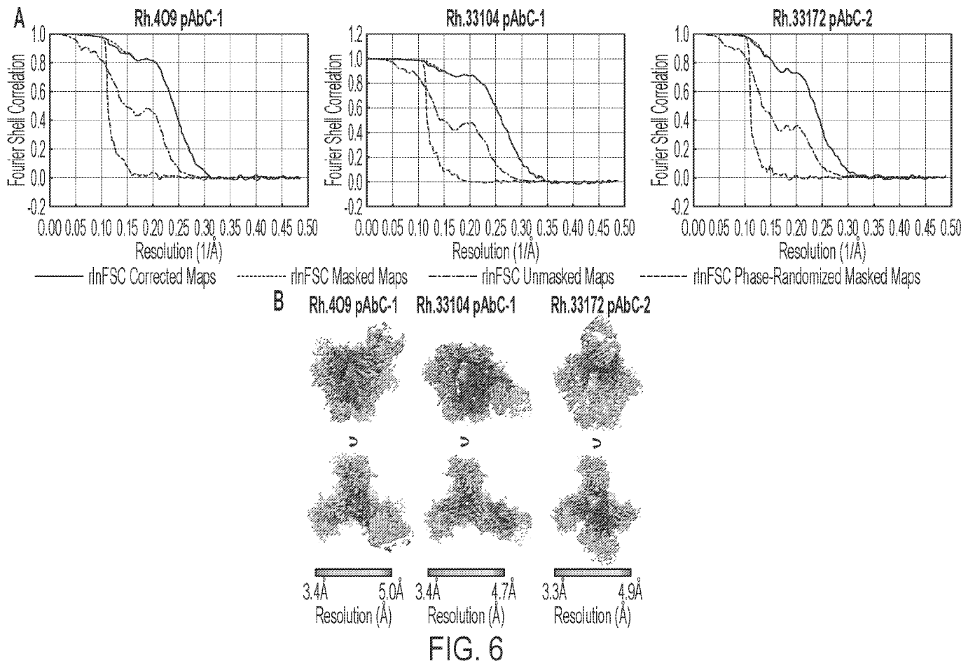

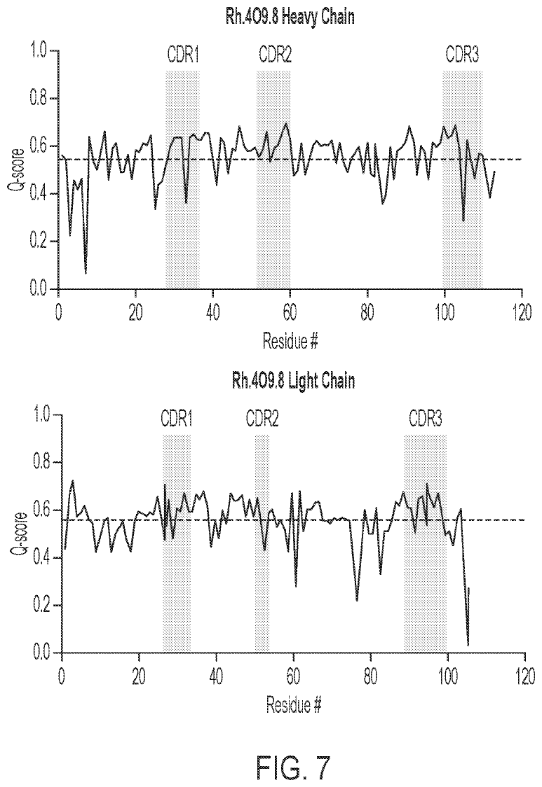

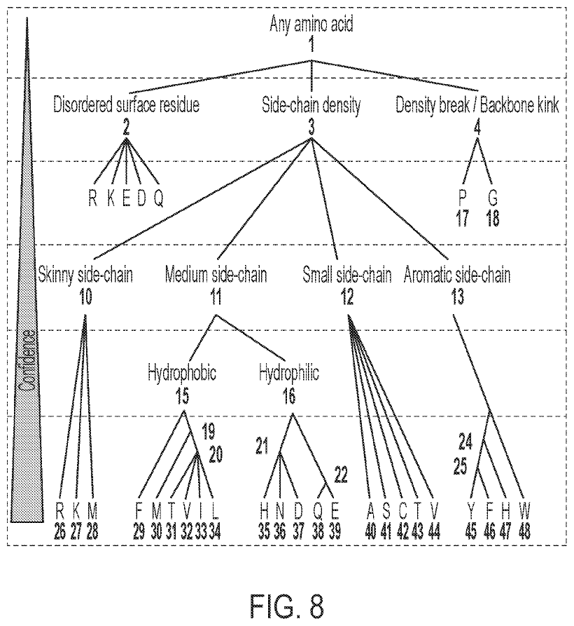

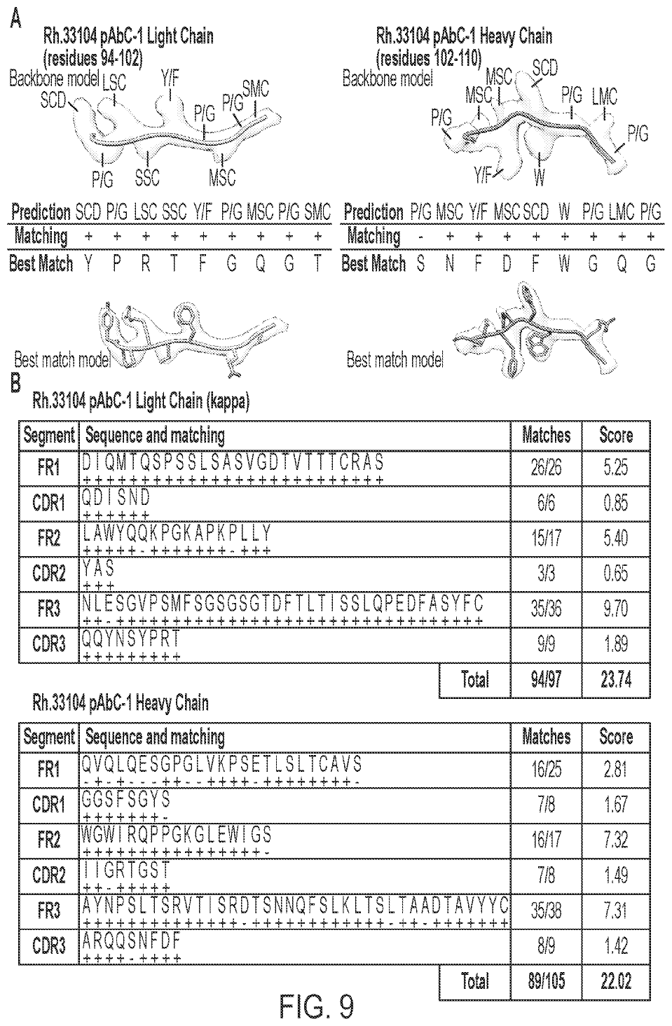

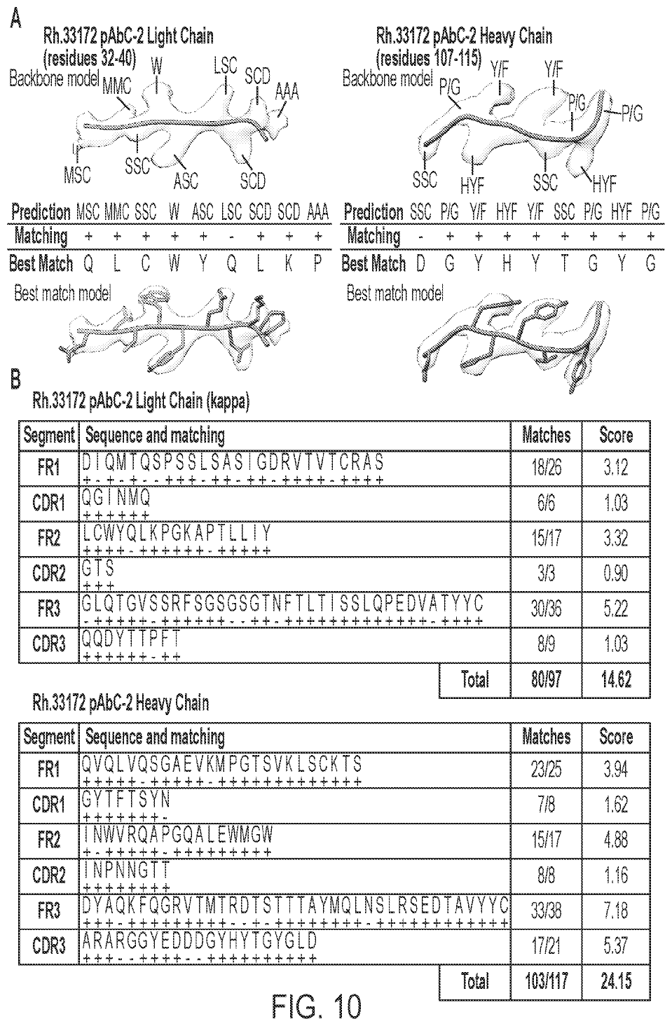

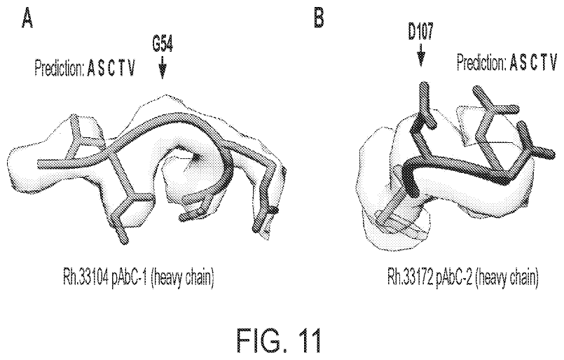

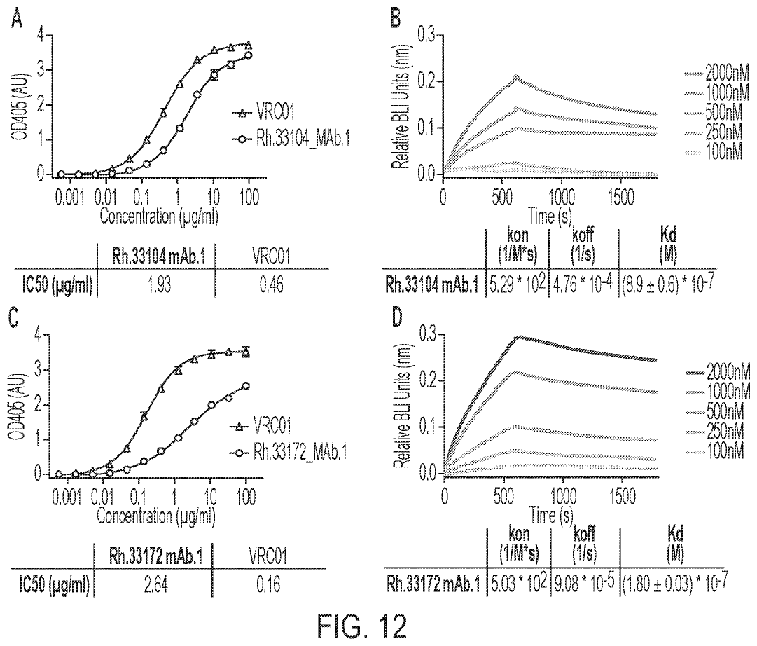

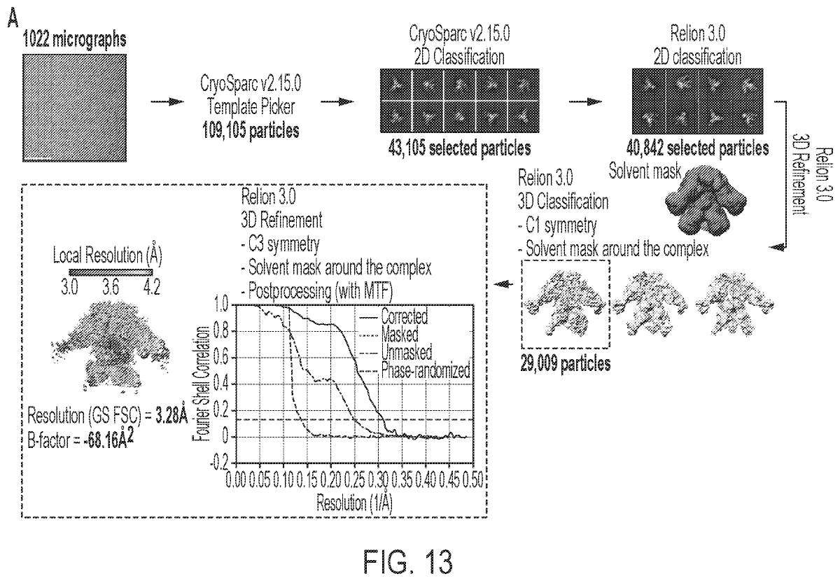

Next, the inventors sought to identify clonal relatives for Rh.33104 mAb.1 and Rh.33172 mAb.1 antibodies and compare their sequences (in the context of structural data). One of the major advantages of this approach is that the same NGS data can be used for sequence identification and sequence analysis. Germline antibody gene assignment for heavy and light chain sequences was conducted with IgBLAST using a comprehensive Indian origin RM germline BCR database. IgBLAST results were filtered for productive reads and clustered into clonal lineages using Chango-O and SHazaM. Full sequence alignments of clonal relatives of Rh.33104 mAb.1 and Rh.33172 mAb.1 were analyzed. The diversity of sequences within the CDR regions of aligned heavy and light chains is shown in FIG. 5.

In the case of Rh.33104 mAb.1 minimal CDR sequence diversity was observed. Consensus sequence for the heavy chain was based on several clonally related sequences and was identical to the Rh.33104 mAb.1 (best match sequence) in the CDR regions (FIG. 5. a). Light chain sequences displayed greater diversity within the CDRs, and that was also reflected in the discordance between the best match and the consensus sequences at 4 sites. The analysis of structural data from cryoEMPEM polyclonal maps at these sites revealed that while both sequence variants could fit within the side-chain-corresponding densities at positions 51 and 91, the structural information at positions 32 and 96 corresponded better to the best-match sequence than the consensus. This is not surprising as the best match sequence was selected based on the structural information.

These data exemplify the disconnect that exists between the antibody repertoire analysis on the level of B-cells (based on BCR sequencing) and the polyclonal antibodies actually present in serum. However, our findings illustrate the power of cryoEMPEM to discriminate between small, side-chain-level structural features and show how this information can be used to complement the sequence analysis.

### Example 6: Antigen Expression and Purification

Antigen expression and purification were performed as described previously (Antanasijevic et al., 2020). Briefly, BG505-SOSIP.v5.2(7S) N241/N289 and BG505 SOSIP MD39 construct genes, codon-optimized for mammalian cell expression, were subcloned into a pPPI4 vector. Sanger sequencing was applied to verify the resulting vector sequence. These constructs were expressed in 293F cells (Thermo Fisher Scientific) and purified from the cell supernatant using PGT145 or 2G12 immunoaffinity chromatography. 3M MgCl₂ buffer was used for protein elution from the matrix. BG505 SOSIP samples were concentrated, buffer exchanged to TBS (Alfa Aesar) and subjected to size-exclusion chromatography (SEC). HiLoad 16/600 Superdex pg200 (GE Healthcare) running TBS buffer was used for SEC purification. Fractions corresponding to the BG505 SOSIP antigen were pooled, concentrated to 1 mg/ml and frozen for storage.

The assignment of polyclonal antibody sequences was performed using models and maps from previously published polyclonal datasets (Nogal et. al. 2020; Antanasijevic et al. 2020). Polypeptide backbone of each antibody, represented as poly-alanine pseudo-model, is manually relaxed into the map in Coot and the number of amino-acid residues is adjusted to achieve the most optimal model-to-map fit on the backbone level. The antibody-antigen complex structure was then subjected to automated refinement in Rosetta, with corresponding density map restraints. Sequence assignment the heavy and light chains was performed manually in Coot, using the models and maps from the automated refinement step. We developed a system for amino-acid assignment based on the corresponding structural features, that also takes into consideration the degree of certainty associated with each assignment. Density volume surrounding each amino acid is attributed a hierarchical category identifier representing a predefined subset of amino acid residues that best correspond to the density. In the final output, the heavy and light chain sequences are represented as strings of numerical category identifiers. In addition to shape properties, our system allows to further categorize certain amino acid groups (e.g. medium side-chain group) based on the local environment (i.e. hydrophobic/hydrophilic) which helps narrow down the list of possible amino acids.

### Example 7: Sequence Alignment and Scoring

Sequence match searching was performed using Python 3.6.3 in a Jupyter Notebook environment. To prepare for alignment, the hierarchical ambiguity codes from FIG. 3A were first translated into numerical codes and organized into a recursive binary tree. This allows the algorithm to call upon any specific branch and fetch all downstream amino acid possibilities in an efficient manner. The search can be performed on two main data formats: protein FASTA files and .tsv formatted output from igblastn results. The search can also compare the ambiguity codes directly to the allele database present in the Indian Origin Rhesus macaque Germ Line Database (GLD) currently hosted by the lab. Search results against FASTA files and the GLD are basic and do not return information outside of alignment of the main section of sequence. Searches run on .tsv files from igblastn (referenced below as “data frame”) allow the user to pass queries in a split method based on (BLAH) designations for relevant framework and CDR loop regions. For the main searches featured here, query strings of ambiguity codes were manually segmented into the following regions: FW1, CDR1, FW2, CDR2, FW3, CDR3. Before searching, the data frame to be searched is subjected to filtering based on user-defined criteria. The user can filter out entries from the data frame based on any column that exists, typically restricting based on a range of desired lengths of FW and CDR regions. Once the data frame is prepared and a search is initiated, the algorithm exhaustively compares each query segment to the relevant column from the data frame in pairwise fashion. Alignment is allowed to shift by a factor defined by the user (default 2 AA), and results are returned based on a maximum alignment score for each query to each subject sequence. Scores are calculated as the inverse of how many amino acids are represented by each ambiguity code at each position if there is a match. For example, if an ambiguity code represents a branch that has 5 possible amino acids and there is a match, the position is assessed a score of 1/5, or 0.2. Scores range from 1/20 (0.05) to 1/1 (1.00). Scores are tallied for each query section of each row of the data frame and returned to the user in csv format for easy manipulation and selection of high scoring alignments.

### Example 8: Analysis of Sequence Alignment Results

For search result analysis, we calculated the alignment scores for the entire sequence (Total Score) and the complementarity-determining regions (CDR-only Score). The heavy and light chain sequences featuring a combination of the highest Total and CDR-only scores from each search were selected for subcloning and expression. For analysis of the light chain alignment results, we have also calculated the mean total alignment scores for all NGS sequences in the corresponding Ig-k and Ig-l queries. The comparison of maximum and mean total alignment scores from the two queries was applied to determine if the antibody light chain was Ig-k or Ig-l.

### Example 9: Antibody Expression and Purification

Top-scoring heavy and light chain sequences from the corresponding NGS database searches were subcloned into the AbVec-hIgG1 and AbVec-hIgKappa expression vectors, respectively. Sanger sequencing was applied to verify the final DNA vectors. 500 μg of the heavy chain and 250 μg of the light chain DNA expression vectors were applied for co-transfection of 1 L of HEK293F cells to produce Rh.33104 mAb.1 and Rh.33172 mAb.1 (as full IgG and Fab fragments). PEI MAX (Polysciences, Inc.) was used as a transfection reagent at three-fold mass excess with respect to total DNA amount. Antibodies were purified from cell supernatants using the MAbSelect Xtra (GE Healthcare Life Sciences) and CaptureSelect IgG-CH1 (ThermoFisher Scientific) columns for IgG and Fab purification respectively. Antibody samples were concentrated, buffer exchanged to TBS buffer (Alfa Aesar), and then subjected to SEC purification (HiLoad 16/600 Superdex S200 pg column; GE Healthcare Life Sciences).

### Example 10: Sandwich ELISA Assays

Sandwich ELISA experiments were performed with Rh.33104 mAb.1 and Rh.33172 mAb.1 (as IgG). All experiments were performed in triplicates. BioStack Microplate Stacker system (BioTek) was used for buffer addition and wash steps. 12N antibody with specificity towards the base of BG505 SOSIP, was diluted to 3 μg/ml and immobilized onto high-binding, 96-well microplates (Greiner Bio-One) for 2 hours at room temperature. The plates were washed 3 times with TB ST buffer (TBS+0.1% Tween-20) and blocked overnight with TBS+5% bovine serum albumin (BSA)+0.05% Tween-20 at 4° C. Plates were washed 3 times with TBST, followed by the addition of the antigen solution (PBS+1% BSA+3 μg/ml of BG505 SOSIP). For Rh.33172 mAb.1 experiments we used BG505 SOSIP.v5.2(7S) N241/N289 construct; for Rh.33104 mAb.1 experiments we used BG505 SOSIP MD39. This was done to match the BG505 SOSIP construct to the original immunogen that elicited the corresponding polyclonal antibodies in rhesus macaques (Antanasijevic et al., 2020). The plates were incubated with antigen solution for 2 hours and then washed 3 times with TBST. Serial 3-fold dilutions of Rh.33104 mAb.1 or Rh.33172 mAb.1 (starting at 100 μg/ml) were prepared in TBS and added to plates coated with corresponding antigen. The plates were incubated for 2 hours at room temperature and subsequently washed 3 times with TB ST. AP-conjugated AffiniPure goat anti-human IgG, (Jackson Immunoresearch, Cat #109-055-097) was diluted 1:4000 in TBS+1% BSA buffer and added to each well for 1 hour at room temperature. Following 3 wash steps with TBST, 1-Step PNPP Substrate Solution (Thermo-Fisher Scientific) was applied to each well for detection. Synergy H1 plate reader (BioTek) was used for acquisition of colorimetric data by recording the absorbance at 405 nm wavelength. Data was analyzed in Graphpad Prism software and midpoint titers were determined.

### Example 11: Biolayer Interferometry (BLI)

Octet Red96 instrument (FortéBio) was used for BLI data collection. Antibody and antigen solutions were prepared in kinetics buffer (DPBS+0.1% [w/v] BSA+0.02% [v/v] Tween-20). All BLI experiments were conducted at 25° C. BI I experiments with IgGs were performed as described in Ozorowski et al., 2018; Antanasijevic et al., 2020. Rh.33104 mAb.1, Rh.33172 mAb.1 and VRC01 antibodies (as IgGs) were diluted to 5 μg/ml and immobilized onto human IgG Fc capture (AHC) biosensors (FortéBio). VRC01 served as a positive control. Antibody-coated sensors were then transferred to wells with corresponding BG505 SOSIP antigens (see ELISA method section above for explanation) at 1000 nM concentration. Association and dissociation steps were set to 180 s and 300 s, respectively. Data was processed using Octet System Data Analysis v9.0 (FortéBio). Negative control measurements (with kinetics buffer) were used for background correction. Final plots were prepared in Graphpad Prism.

Experiments with monoclonal antibodies (as Fabs) were performed as described previously (Cottrell et al., 2020). Fabs were diluted to 25 μg/ml and immobilized onto anti-human Fab-CH1 (FAB2G) biosensors (FortéBio). Serial 2-fold dilutions of the corresponding BG505 SOSIP antigens (see ELISA method section above for explanation) were prepared for binding studies, starting at 2000 nM. The lengths of association and dissociation steps were set to 600 and 1200s, respectively. Data processing and determination of kinetic parameters were performed in Octet System Data Analysis v9.0 software (FortéBio). Data plots were prepared in Graphpad Prism.

### Example 12: CryoEM Analysis of Monoclonal Antibody Complexes—Preparation of Complexes

Rh.33104 mAb.1 complex preparation: 250 μg of BG505 SOSIP MD39 was incubated with 600 μg of Rh.33104 mAb.1 Fab and 600 μg of RM20A3 Fab (Cottrell et al., 2020; Berndsen et al., 2020), at room temperature, overnight. The complex was SEC-purified using a HiLoad 16/600 Superdex pg200 (GE Healthcare) column, with TBS as a running buffer. SEC fractions corresponding to the complex were pooled and concentrated to 6.0 mg/ml using an Amicon filter unit with 10 kDa cutoff (EMD Millipore).

Rh.33172 mAb.1 complex preparation: 250 μg of BG505 SOSIP.v5.2(7S) N241/N289 was incubated with 600 μg of Rh.33172 mAb.1 Fab and 600 μg of RM19R Fab (Cottrell et al., 2020), at room temperature, overnight. All other purification steps were equivalent as with Rh.33104 mAb.1 complex.

### Example 13: CryoEM Analysis of Monoclonal Antibody Complexes—Grid Preparation

UltrAuFoil R 1.2/1.3 grids (Au, 300-mesh; Quantifoil Micro Tools GmbH) were used for sample vitrification. The grids were treated with Ar/O₂ plasma (Solarus 950 plasma cleaner, Gatan) for 10 s immediately prior to sample application. 0.5 μL of 0.04 mM lauryl maltose neopentyl glycol (LMNG) stock solution was mixed with 3.5 μL of the complex and 3 μl were immediately loaded onto the grid. Grids were prepared using Vitrobot mark IV (Thermo Fisher Scientific). Temperature inside the chamber was maintained at 10° C. while humidity was at 100%. Blotting force was set to 0, wait-time to 10 s while the blotting time was varied within a 4-7 s range. Following the blotting step, the grids were plunge-frozen into liquid ethane, cooled by liquid nitrogen.

### Example 14: CryoEM Analysis of Monoclonal Antibody Complexes—Data Collection and Processing

Samples were imaged on FEI Titan Krios electron microscope (ThermoFisher) operating at 300 keV. The microscope was equipped with the K2 summit detector (Gatan) operating in counting mode. Exposure magnification was 29,000 and the pixel size was 1.03 Å (at the specimen plane). Leginon software suite was used for automated data collection. Data collection information for the two datasets featuring different monoclonal antibody complexes are presented. Micrograph movie frames were aligned and dose-weighted using MotionCor2 and GCTF was applied for estimation of CTF parameters. Initial processing steps (particle picking and 2D classification) were performed in cryoSPARC.v2. Ab initio refinement in cryoSPARC was applied to generate the initial reference model for each complex. Clean particle stack was subsequently transferred to Relion/3.0 for further 2D and 3D processing steps.

### Example 15: CryoEM Analysis of Monoclonal Antibody Complexes—Model Building and Refinement

Postprocessed cryoEM maps from Relion/3.0 were used to generate atomic models. PDB entry 6vfl was used as initial model for BG505 SOSIP-corresponding part of the complex. The sequence was adjusted to match the exact BG505 SOSIP variant used for the preparation of imaged monoclonal antibody complex (BG505 SOSIP MD39 or BG505 SOSIP.v5.2(7S) N241/N289). Initial models for RM20A3 and RM19R antibodies were adapted from Berndsen et al., 2020 and Cottrell et al., 2020, respectively. ABodyBuilder was applied to create the initial Fab models for Rh.33104 mAb.1 and Rh.33172 mAb.1. Individual components were docked into the corresponding parts of each cryoEM map in UCSF Chimera to create the initial atomic models. Iterative rounds of manual model refinement in Coot and automated refinement using Rosetta were performed to produce the final models. For model validation we applied EMRinger and MolProbity packages. Model refinement statistics were reported. The refined models have been submitted to the Protein Data Bank.

### Example 16: B-Cell Sorting

Nonhuman primate immunizations: Rhesus macaques were immunized at weeks 0, 8, and 24. Immunization were administered subcutaneously, divided between the right and left mid-thighs. Animals were immunized with BG505 SOSIP MD39 trimer (100 ug total) or BG505v5.2.st.N241+N289 T33-31-NP nanoparticle (128.6 ug total) with Matrix-M (75 U total). Lymph node fine needle aspirates were performed as previously described (Cirelli et al.) at weeks 8, 11, 14, and 27. Lymph node biopsies were performed between weeks 40 and 42.

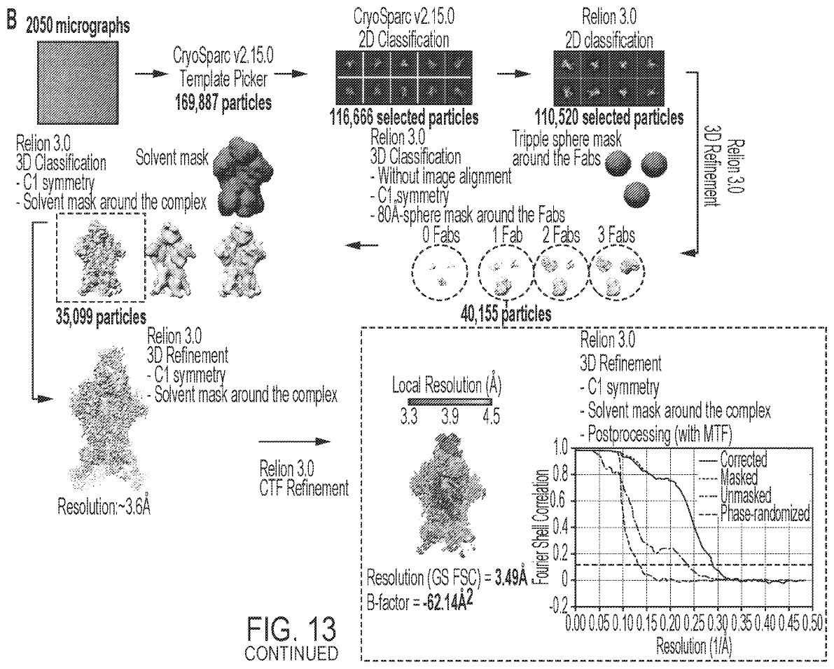

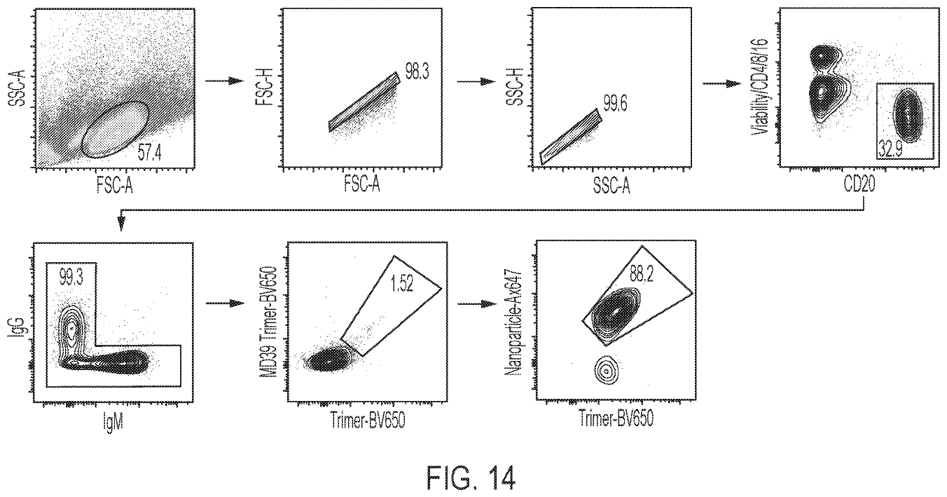

B-cell sorting: B cell sorting procedure is illustrated in FIG. 14, and also described below. Biotinylated BG505 SOSIP MD39 trimer and BG505v5.2 trimer were generated as previously described (Cireli et al. 2019). Biotinylated proteins were individually premixed with fluorochrome-conjugated streptavidin (Brilliant Violet 650 or Brilliant Violet 421, BioLegend) at RT for 20 minutes. BG505 SOSIP MD39-ferritin and BG505v5.2.st.N241+N289 T33-31 NP nanoparticles were generated and directly conjugated to Alexa Fluor 647 (Thermo Fisher). Cells were incubated with indicated probes for 30 minutes at 4° C., washed twice and then incubated with surface antibodies for 30 minutes at 4° C. Cells were washed and then sorted on a FACSAria II.

Rh.33104 (BG505 SOSIP MD39 immunized)—LN cells were stained with the following probes and antibodies: MD39 trimer-Brilliant Violet 650, MD39 trimer-Brilliant Violet 421, MD39-Ferritin nanoparticle-Alexa Fluor 647, fixable viability dye-eFluor 780 (Thermo Fisher), mouse anti-human CD4 APC eFluor 780 (SK3, Thermo Fisher), mouse anti-human CD8 APC eFluor 780 (RPA-T8, Thermo Fisher), mouse anti-human CD16 APC eFluor 780 (ebioCB16, Thermo Fisher), mouse anti-human CD20 Alexa Fluor 488 (2H7, BioLegend), mouse anti-human IgG PE-Cy7 (G18-145, BD Biosciences), mouse anti-human IgM PerCP-Cy5.5 (G20-127, BD Biosciences), mouse anti-human CD38 PE (OKT, NHP Reagents), mouse anti-human CD71 PE-CF594 (L01.1, BD Biosciences).

Rh.33172 (BG505v5.2.st.N241+N289 T33-31-NP immunized)—LN cells were stained with the following probes and antibodies: MD39 trimer-Brilliant Violet 650, BG505v5.2 trimer-Brilliant Violet 421, BG505v5.2.st.N241+N289 T33-31NP nanoparticle-Alexa Fluor 647, fixable viability dye-eFluor 780 (Thermo Fisher), mouse anti-human CD4 APC eFluor 780 (SK3, Thermo Fisher), mouse anti-human CD8 APC eFluor 780 (RPA-T8, Thermo Fisher), mouse anti-human CD16 APC eFluor 780 (ebioCB16, Thermo Fisher), mouse anti-human CD20 Alexa Fluor 488 (2H7, BioLegend), mouse anti-human IgG PE-Cy7 (G18-145, BD Biosciences), mouse anti-human IgM PerCP-Cy5.5 (G20-127, BD Biosciences), mouse anti-human CD38 PE (OKT, NHP Reagents), mouse anti-human CD71 PE-CF594 (L01.1, BD Biosciences).

### Example 17: Structural Mapping of Antibody Landscapes to Human Betacoronavirus Spike Proteins

The epitope mapping methods of polyclonal antibodies as disclosed herein may be used against any type of virus. As non-limiting examples, the inventors have shown examples of applying cryoEMPEM and sequencing to determine structural mapping of HIV viruses, flu viruses, coronaviruses, enteroviruses, and isohedral viruses.

The applicability of cryoEMPEM methods for epitope mapping in beta-coronavirus spike proteins in human sera is disclosed in Bangaru et al “Structural mapping of antibody landscapes to human betacoronavirus spike proteins”, bioRxiv 2021.09.30.462459 (available at https://www.biorxiv.org/content/10.1101/2021.09.30.462459v1) which is incorporated by reference in its entirety. The cryoEM polyclonal epitope mapping of flu virus H5N1 is disclosed in Julianna Han, et al “Polyclonal epitope mapping reveals temporal dynamics and diversity of human antibody responses to H5N1 vaccination”, Cell Reports, Volume 34, Issue 4, 2021, which is incorporated by reference in its entirety. Application of electron-microscopy-based polyclonal epitope mapping (EMPEM) to non-polio enteroviruses (NPEVs) is disclosed in Antanasijevic et al, “High-resolution structural analysis of enterovirus-reactive polyclonal antibodies in complex with whole virions”, bioRxiv 2022.01.31.478566 (available at https://www.biorxiv.org/content/10.1101/2022.01.31.478566v1) which is incorporated by reference in its entirety.

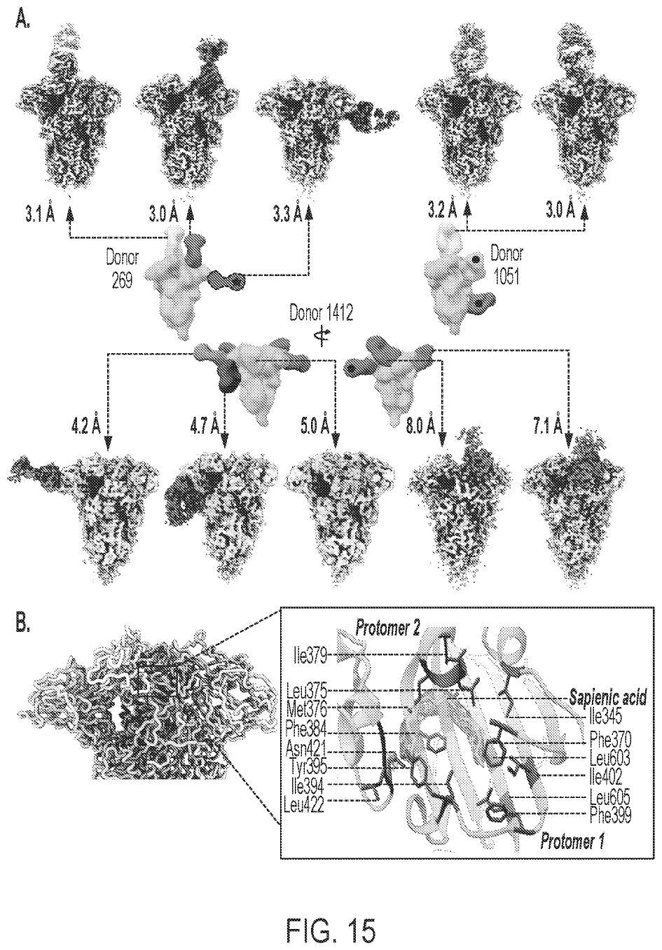

CryoEMPEM analysis of OC43 spike-polyclonal Fab complexes is shown in FIG. 15. (A) illustrates high resolution cryo-EMPEM reconstructions of OC43 spike complexed with polyclonal Fabs derived from sera from HDs 269 (top-left), 1051 (top-right) or 1412 (bottom); the representative composite figures from ns-EMPEM from these donors are shown in the middle. Each map depicts a structurally unique polyclonal antibody class reconstructed at the indicated resolution. OC43 spike is represented in light gray. Fabs marked with a black dot were observed by ns-EMPEM but were not detected by cryo-EMPEM. Fab class from donor 269 marked with a star was resolved by cryo-EMPEM but not by nsEMPEM. (B) Sapienic acid (aquamarine) binding within a hydrophobic pocket in the CTD-CTD inter-protomeric interface. Protomers are colored in light pink, blue or wheat and the interacting residues are shown in gray.

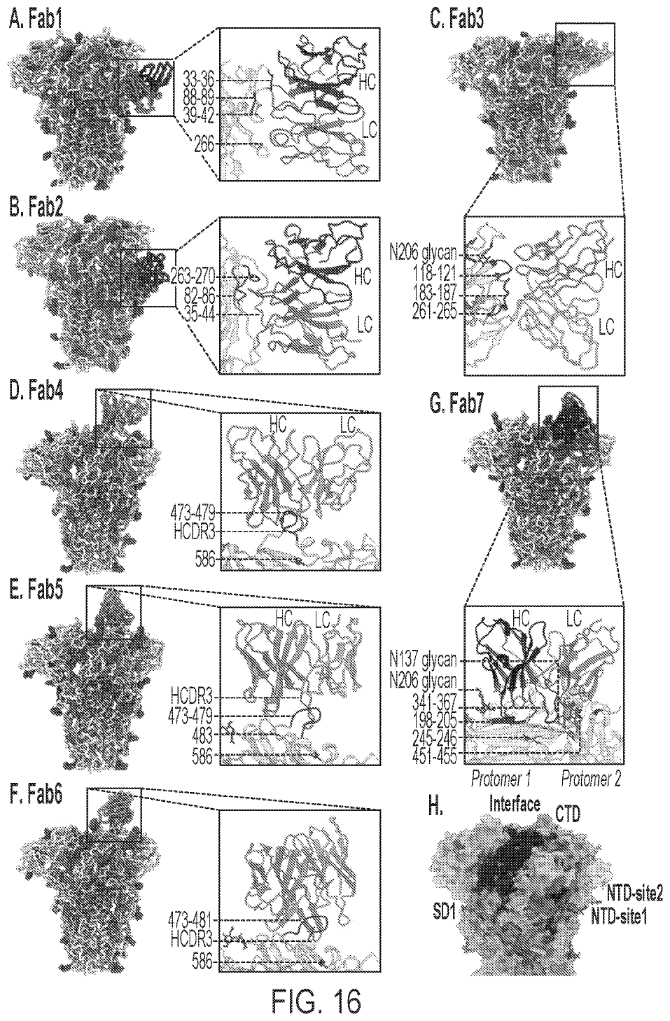

Cryo-EM structures of polyclonal Fabs targeting the OC43 spike (A-G) Tube or ribbon representation of atomic models of OC43 spike-Fab complexes along with zoomed-in views of epitope-paratope interactions are shown in FIG. 16. (A-B) Fab1 and Fab2 (red) target the NTD-site1 or RBS, (C) Fab3 (orange) targets NTD-site2 adjacent to RBS, (D-F) Fab4, Fab5 and Fab6 (yellow) target the CTD and (G) Fab? (blue) targets the NTD-CTD interface. The spike protomers are shown in light blue, light pink or wheat (ribbon representation) with glycans in teal (sphere atom representation) and primary epitope contacts in gray. (H) Surface representation of OC43 spike (gray) showing collective epitopes of Fab1 to Fab10 colored based on their binding site

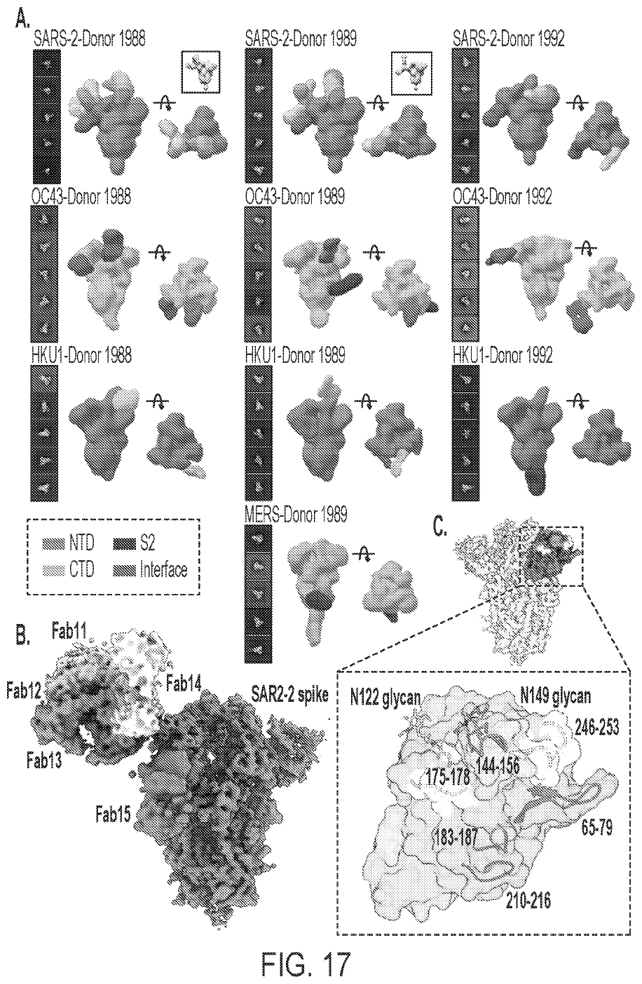

Ns- and cryo-EMPEM analysis of polyclonal Fabs from SARS-2 CD sera are shown in FIG. 17. (A) Representative 2D classes and side and top views of composite figures from ns-EMPEM analysis of polyclonal Fabs from 3 SARS-2 donors complexed with β-CoV spikes. The donor numbers along with the corresponding CoV spikes are indicated above each panel in (A). The Fabs are color-coded based on their epitope specificities as indicated at the bottom-left. SARS-2, OC43, HKU1 and MERS spikes are represented in slate gray, light gray, dark gray and beige respectively. 3D reconstructions displaying potential self-reactive antibodies are shown in grey on the top right corners for both donors 1988 and donor 1999 in complex with SARS-2 spike (B) Composite figure showing 5 unique antibody classes, Fab11 to Fab15 colored in shades of red, to SARS-2 spike NTD reconstructed using cryo-EMPEM analysis of polyclonal Fabs from donors 1988 and 1989 complexed with SARS-2 stabilized spikes. (C) Surface representation of SARS-2 spike showing epitopes of Fabs 11 to 15 from (B) on a single NTD (slate gray) with a zoomed-in view displaying the loop residues comprising each epitope. Loop 144 to 156 with the N149 glycan forms an immunodominant element commonly targeted by Fabs 11 to 14. The sub-epitope colors correspond to each Fab shown in (B).

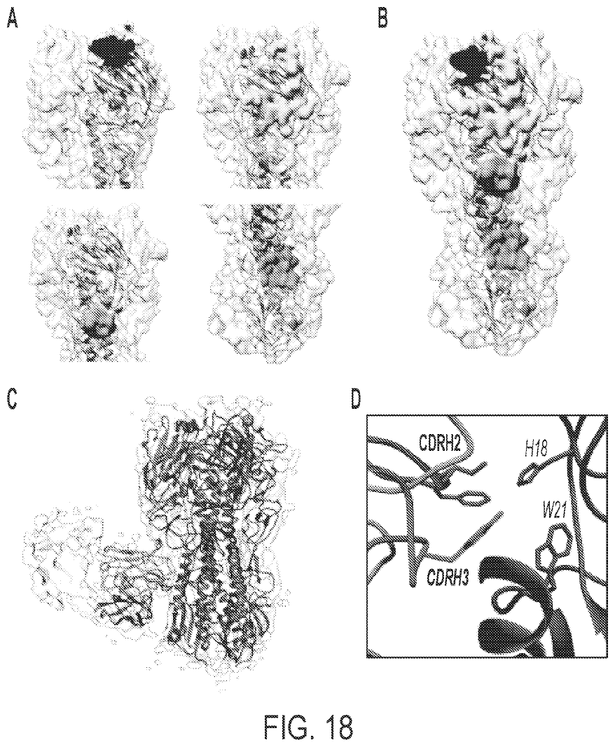

FIG. 18 shows that high-resolution cryoEM defines broadly reactive epitopes of H5N1 vaccine-elicited pAbs. (A) CryoEM-mapped epitopes targeted by pAbs from subject 4 at day 28. RBS epitope, red; lateral patch epitope, yellow; vestigial esterase epitope, pink; VH1-69 epitope, blue. (B) Each epitope specificity marked together on a single trimer. (C and D) CryoEM map of stem immune complex from subject 4 at day 28 with ribbon diagrams for H5 HA (A/Indonesia/5/2005, gray; PDB: 4K62) and mAb 1C01 from subject 4 day 7 docked into the EM density. Full-length side view (C) and zoomed in view of the epitope-paratope interaction (D). V_{H}, blue; V_{L}, green; CDRH2, dark blue; CDRH3, purple; HA residues H18 and W21, red.

Furthermore, we have introduced a modified workflow allowing to image and resolve polyclonal antibodies bound to enteroviruses and other icosahedral viruses, using immune complexes with whole virions (as oppose to recombinant antigens).

EMPEM was performed on immune complexes featuring enteroviruses, specifically CV-A21 viral particles and CV-A21-specific pAbs isolated from mice. The experiments and results are discussed in detail at is disclosed in Antanasijevic et al, “High-resolution structural analysis of enterovirus-reactive polyclonal antibodies in complex with whole virions”, bioRxiv 2022.01.31.478566, which is incorporated by reference herein. Notably, this is the first example of structural analysis of polyclonal immune complexes comprising whole virions. We introduce a data processing workflow that allows reconstruction of different pAbs at near-atomic resolution. The analysis resulted in identification of several antibodies targeting two immunodominant epitopes, near the 3-fold and 5-fold axis of symmetry: the latter overlapping with the receptor binding site. These results demonstrate that EMPEM can be applied to map broad-spectrum polyclonal immune responses against intact virions and define potentially cross-reactive epitopes.

### Example 18: Discovery of Epitope-Specific Functional Antibodies Against Membrane Proteins

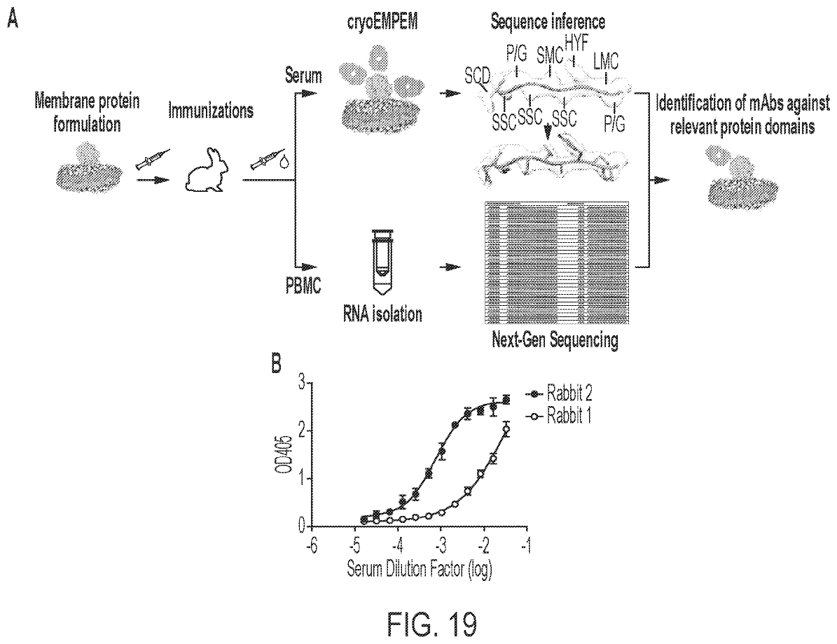

One aspect of the present disclosure of cryoEMPEM method is to identify polyclonal antibodies that are bound to specific epitopes on the surface of ion channels and other membrane proteins. Polyclonal antibodies can be produced in a number of animal models (e.g., rabbits, mice, macaques) through immunizations with recombinantly expressed membrane protein of interest. Membrane proteins are typically stabilized by detergents, lipid nanodiscs or peptidiscs. Antigen-specific antibodies are purified from plasma/serum samples collected from immunized animals, cleaved into Fab fragments and complexed with the corresponding membrane protein(s). The samples are then subjected to cryoEM imaging. Using our focused classification approach, we can resolve polyclonal antibodies bound to different epitopes within the antigen. Structural data allows us to select antibodies that target sites of special importance (e.g., conformation-specific antibodies, and antibodies capable of altering function). Through the application of our method for structure-based sequence determination we can then combine structural data with the B-cell receptor repertoires in the corresponding animal (collected by Next-Generation Sequencing) to identify the monoclonal antibodies belonging to the family of polyclonal antibodies we detected using cryoEM. The workflow is presented in FIG. 19A. This method can lead to the development of therapeutic antibodies as well as antibodies that can be used for detection of membrane proteins by immuno-staining.

For our pilot study we selected 8 membrane-protein candidates: Piezol (human), multidrug resistance protein 1 (P. falciparum), Otop1 (human), Otop1 (zebrafish), solute carrier 27A1 (human), solute carrier 15A4 (human), transmembrane protein 63B (human), transmembrane protein 63B (falcon), formulated in detergent, nanodiscs or peptidiscs. These proteins were injected into New Zealand white rabbits at 4 time points (week 0, 4, 12, 18). Alum was the adjuvant used for all immunizations. Plasma samples were collected biweekly, concluding with week 20, and PBMC samples were collected at week 20.

These experiments are currently in progress, but we have already used enzyme-linked immunosorbent assays (ELISA) to verify elicitation of antigen-specific antibodies for all immunized animals. The examples of ELISA data are shown in FIG. 1B. We are currently pursuing cryoEMPEM and NGS experiments.

### Example 19: Determination of Antibody Germline Sequences Using cryoEMPEM

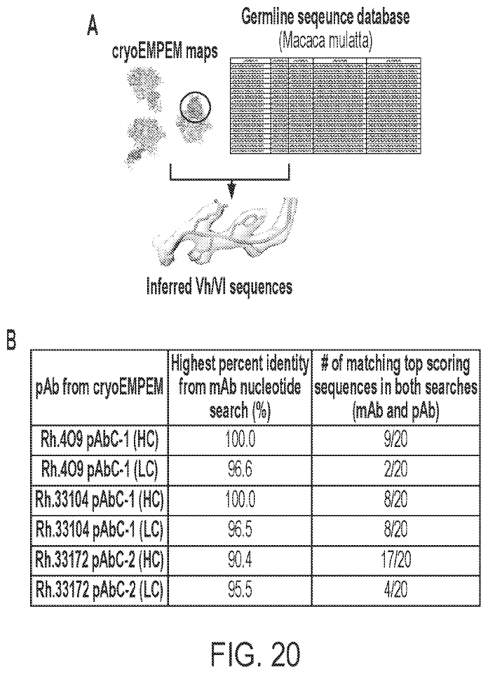

We have adapted our method for determining antibody sequences from structure to be applied for identification of antibody germline genes. The original method was based on using B-cell receptor (BCR) sequences from the same animal or human subject and the same time point at which polyclonal plasma/serum sample was collected. While B-cell receptor sequences are highly variable within each individual and continuously evolve, the germline precursor sequences of V, D and J genes are constant and shared between individuals. Therefore, we adapted the current approach for sequence determination to identify the germline V, D and J genes of the heavy chain and V and J genes of the light chain for the polyclonal antibody of interest reconstructed during cryoEMPEM analysis (FIG. 20A). This method can work with polyclonal antibody samples from any species (e.g., rabbit, mouse, macaque, human), but the germline sequence database needs to be species matched. This information can be used for analysis of vaccine-elicited polyclonal antibodies or for antibody design.

For proof of concept, we tested the applicability of this method using polyclonal antibodies recovered from rhesus macaques immunized with HIV Env immunogens. Specifically, we used cryoEMPEM maps from our previous work (Antanasijevic, Bowman et al., Sci Adv, 2022) and a rhesus macaque germline database. These datasets were selected because they featured (1) high resolution maps with polyclonal antibodies, and (2) monoclonal antibodies belonging to these polyclonal families have been isolated and validated. Therefore, we were able to compare if our method for determination of germline sequences using structural cryoEMPEM data, would match the traditional method for germline inference based on alignment of mAb nucleotide sequences.

For the 3 antibodies that were tested, Rh.33104 pAbC-1, Rh.4O9 pAbC-1 and Rh.33172 pAbC-2, we found that top-scoring Vh/Vl germline sequences identified by the structure-based approach shared >95% sequence identity to the top-scoring germline Vh/Vl sequences identified by traditional method using corresponding monoclonal antibodies (Rh.33104_mAb1, Rh.4O9.8 and Rh.33172_mAb1, respectively). In 2/6 cases the top-scoring V gene hit was identical in both methods (FIG. 20B). This data confirms that our approach can be used to reliably identify germline precursor sequences of polyclonal antibodies observed by cryoEMPEM.

### Example 20: In Silico Method for Antibody Design Using Structural Data from cryoEMPEM Maps

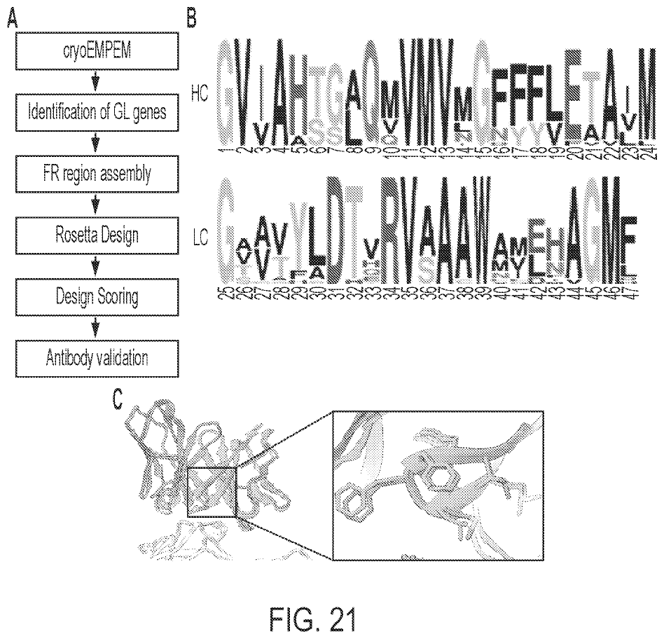

The existing approach for determining antibody sequences from structure, relies on two inputs (1) high-resolution structural data from cryoEMPEM and (2) B-cell receptor database. The latter requires collecting PBMC samples from immunized animals and subjecting the B-cells to next-generation sequencing; the efficacy of which can vary greatly. In order to bypass the need for BCR database in antibody sequence determination, we developed a method based on Rosetta to engineer antibodies directly from cryoEMPEM maps. The method consists of (1) identifying V and J germline genes for polyclonal antibody reconstructed by cryoEMPEM (as described in the previous section), (2) using the germline heavy and light chain sequences to assemble antibody framework regions, (3) applying Rosetta design to build the CDR loops in the heavy and light chain of the antibody, and (4) applying custom-designed algorithms to score models designed by Rosetta to identify the highest affinity antibodies. This workflow is illustrated in FIG. 21A.

To validate the workflow, we used the Rh.4O9 pAbC-1 map generated by cryoEMPEM. Rosetta design produced 1000 models based on the structural restraints from the cryoEMPEM map. The sequence logo for Rosetta models is presented in FIG. 21B. We compared the top-scoring models to the sequence of Rh.4O9.8, a monoclonal antibody isolated from the same animal that we have previously shown is a clonal relative of Rh.4O9 pAbC-1 family. On the level of backbone, we observed excellent agreement between Rh.4O9.8 and top-scoring antibody designed by Rosetta. Additionally, HCDR3 loops shared highly similar sequences and conformations between the two, indicating that our in silico antibody design method can produce antibodies comparable to the ones produced in vivo. The kinetic properties of designed antibodies may also be determined by the method disclosed herein.

This method can be used to design antibodies for therapeutic and research purposes. Additionally, through further redesign we may be able to achieve higher affinities compared to naturally-produced antibodies.

### Example 21: Application of cryoEMPEM for Isolation and Identification of Auto-Immune Antibodies

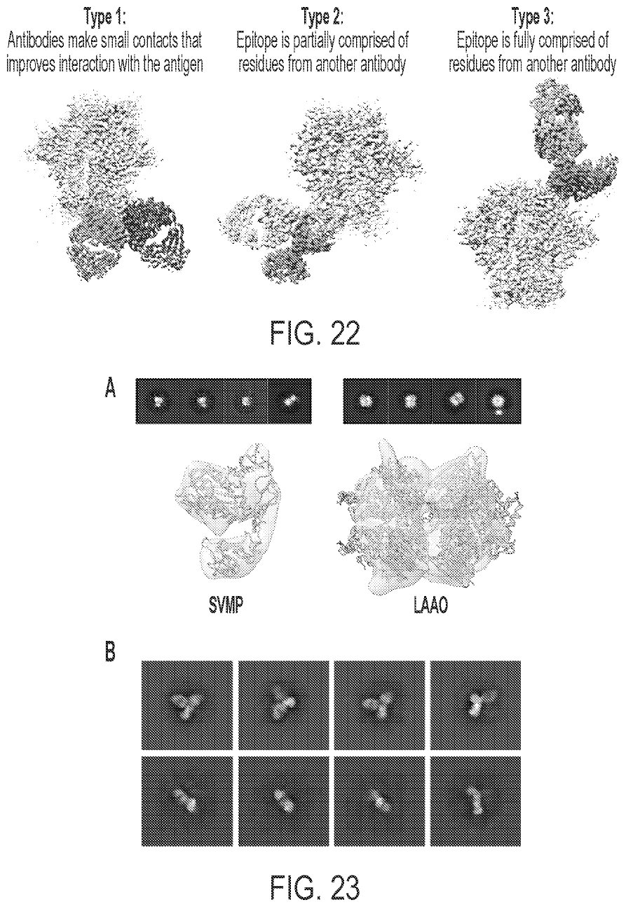

Using cryoEMPEM, we have identified a new type of polyclonal antibodies for which the epitopes partially consist of other antibodies. This was first observed in rhesus macaques following several immunizations with HIV Env trimer immunogens but was subsequently shown in rabbits and humans as well. High-density glycan shield largely restricts access to epitopes on the surface of HIV Env that can be accessed by antibodies. This results in saturation of available epitopes by polyclonal antibodies elicited after the first few doses of immunogen. With additional immunizations, novel antibodies start to get elicited against neo-epitopes partially comprising other antibodies pre-bound to the antigen. We have identified 3 distinct types of these antibodies, differing in how they interact with the antigen and other antigen-bound antibodies (FIG. 22).

This is a novel observation that may have important consequences for vaccine design, particularly when it comes to the maximum number of immunogen doses that should be administered. Induction of type-3 antibodies can lead to depletion of other antibodies and cause immune disorders. We are continuing to explore this novel class of antibodies.

Importantly, these antibodies cannot be isolated by traditional methods like B-cell sorting because they require other polyclonal antibodies to be pre-bound to the antigen. However, we can readily reconstruct them to high resolution through cryoEMPEM and determine their sequence using our sequence-from-structure method.

### Example 22: Application of cryoEMPEM for Characterization of Antibodies Against Snake Venom Proteins

Snake venom consists of a variety of protein components that trigger different types of pathways after they reach the bloodstream. Antivenoms are polyclonal antibodies typically isolated from sheep or horses that target different protein components in venom. We applied electron microscopy to characterize the protein components of Echi Ocellatus venom. Despite great heterogeneity that exists in the sample we were able to identify several protein species based on their structural features (FIG. 23A). We then combined the venom with antivenom antibodies (EchiTAbG, MicroPharm Ltd) and purified the immune complexes using size exclusion chromatography. The immune complexes were then subjected to cryoEMPEM analysis (FIG. 23B). Using cryoEM we can readily detect antibodies bound to different proteins with clearly discernible high-resolution features. We are currently optimizing our focused classification workflows to obtain 3D reconstructions of these antibodies and their corresponding antigens.

In one aspect, the cryoEMPEM methods disclosed herein allows one to identify (1) venom proteins targeted by antivenom antibodies, (2) epitopes that antivenom antibodies are bound to, and (3) sequences of bound antibodies. We apply cryoEMPEM to comprehensively evaluate antibody responses to different proteins and grade them based on their ability to confer protection against snake venom. That information is then applied to design poly-reactive antivenom cocktails and vaccine candidates capable of eliciting antibodies against a broad range of snake venoms.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the full scope of the present disclosure, and does not pose a limitation on the scope of the invention otherwise claimed. No language in the specification should be construed as indicating any non-claimed element essential to the practice of the claimed invention.

It should be apparent to those skilled in the art that many more modifications besides those already described are possible without departing from the full scope of the concepts disclosed herein. The disclosed subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

1. A method of identifying monoclonal antibodies from polyclonal sera, comprising:
utilizing cryogenic electron microscopic polyclonal epitope mapping (cryoEMPEM) to determine structural information of a plurality of antigen-antibody complexes in the polyclonal sera, to thereby assign the heavy chain (HC) and light chains (LC), and to identify complementarity determining regions (CDRs);
projecting, from cryoEM density maps, sequences of polyclonal serum derived antibodies bound to the antigen;
identifying clonal family members of the projected antibodies, by superimposing cryoEM density maps with sequence and structural data in next generation sequencing (NGS) database;
synthesizing the identified monoclonal antibodies corresponding to the identified clonal family members; and
verifying that the synthesized monoclonal antibodies interact with the antigen in a manner equivalent to the corresponding polyclonal antibodies.

2. The method of claim 1, wherein the clonal family members are further identified by user-defined sequence assignments as a series of numbers.

3. The method of claim 1, wherein enzyme-linked immunosorbent assay (ELISA) and biolayer interferometry (BLI) are used to confirm binding of the antibody to the antigen.

4. The method of claim 1, wherein the resolution of the cryoEMPEM map is about 3-4 Å.

5. The method of claim 1, wherein the identification of clonal family members further comprises categorizing amino acids in the projected antibodies based on common structural features.

6. The method of claim 1, wherein clonal family members are identified in silico by a sequence alignment and scoring program comprising the steps of:
a. receiving HC and LC repertoires from NGS database;
b. receiving CDR/FR segment assignment from structure based sequence assignment;
c. filtering the HC and LC repertoires and CDR/FR sequence assignment based on CDR length;
d. aligning the filtered sequences to NGS sequences; and
e. scoring the aligned sequences based on common structural features.

7. The method of any one of the preceding claims, wherein the identified monoclonal antibodies are for treatment of a disease caused by the antigen.

8-9. (canceled)

10. The method of claim 1, further comprising designing an antibody in silico using structural data from the cryoEM density maps.

11. A method of determining monoclonal antibody sequence from cryoEMPEM maps and NGS data, comprising:
utilizing structural information obtained from cryoEMPEM maps and NGS data of B-cell repertoires to identify the most probable combination of heavy and light chain pairs corresponding to the antibody of interest;
wherein the structural information is obtained from a structure-based sequence assignment algorithm that uses different categories of amino acids at each position in a degenerate fashion; and
wherein the algorithm performs an alignment of the predicted string of amino-acid category identifiers to the antibody sequences from the NGS database and scores the quality of alignment, to thereby select the highest scorers as the monoclonal antibody sequence.

12. The method of claim 11, further comprising synthesizing the monoclonal antibodies

13. The method of claim 11, further comprising binding the synthesized monoclonal antibody with to the antigen to confirm tight binding.

14-15. (canceled)

16. The method of claim 11, wherein the categorization of amino acids is based on common structural features.

17. (canceled)

18. A method of designing a vaccine for a disease, comprising:
obtaining, or having obtained, a sample from the patient;
determining structural information of a plurality of antigen-antibody complexes in the patient sample, via cryoEMPEM, to thereby assign the heavy and light chains and to identify complementarity determining regions;
analyzing cryoEM density maps in silico to predict possible antibody sequences bound to the antigen;
identifying clonal family members of the antibody, by superimposing cryoEM density maps with sequence and structural data in NGS data database; and
designing a vaccine for the disease by synthesizing the monoclonal antibody corresponding to the identified clonal family members.

19. The method of claim 18, wherein the sample is a blood or plasma sample.

20. The method of claim 18, wherein the sample is a tumor sample.

21. The method of claim 18, wherein the disease is a bacterial disease.

22. The method of claim 18, wherein the disease is a viral disease.

23. The method of claim 22, wherein the viral disease is SARS-CoV-2.

24. The method of claim 18, wherein the disease is cancer.

25. The method of claim 18, wherein the clonal family members are further identified by user-defined sequence assignments as a series of numbers.

26-29. (canceled)
